(19) United States
(12) Patent Application Publication
(10) Pub. No.: US 2023/0416410 A1
(43) Pub. Date: Dec. 28, 2023

(54) BOVINE ANTIBODY VARIANTS

(71) Applicant: Zoetis Services LLC, Parsippany, NJ (US)

(72) Inventors: Catherine J. Strietzel, Kalamazoo, MI (US); Sandra Ann Marie Lightle, Kalamazoo, MI (US); Henry Luis Campos, Kalamazoo, MI (US); Yaqi Zhu, Kalamazoo, MI (US); Gregory John Fici, Portage, MI (US); Alexander Pogacsas Ruhland Bally, Fort Collins, CO (US); Prajna SHANBHOGUE, Lafayette, CA (US)

(21) Appl. No.: 18/037,514

(22) PCT Filed: Nov. 19, 2021
(86) PCT No.: PCT/US2021/060161

§ 371 Date: May 17, 2023

Related U.S. Application Data
(60) Provisional application No. 63/116,491, filed on Nov. 20, 2020.

(51) Int. Cl. C07K 16/46; C07K 14/705

(52) U.S. Cl. CPC C07K 16/462; C07K 14/70521; C07K 2317/52; C07K 2317/71; C07K 2317/24; C07K 2319/31

(57) ABSTRACT

The invention relates generally to bovine antibody variants and uses thereof. Specifically, the invention relates to mutations in the constant region of bovine antibody for improving various characteristics.

### CROSS-REFERENCE TO RELATED APPLICATIONS

This application claims priority to and the benefit of U.S. Provisional Patent Application 63/116,491, filed Nov. 20, 2020, which is incorporated by reference herein in its entirety.

### FIELD OF THE INVENTION

The invention relates generally to bovine antibody variants and uses thereof. Specifically, the invention relates to one or more mutations in the Fc constant region of bovine antibody for improving various characteristics.

### BACKGROUND OF THE INVENTION

Bovine IgG monoclonal antibodies (mAbs) can be effective therapeutics in veterinary medicine. Several years ago, three bovine IgG subclasses were identified. However, not much work has been done to improve the chraceteristics of bovine IgGs.

Through a recycling mechanism, the neonatal Fc receptor (FcRn) prolongs the half-life of an IgG in a pH-dependent interaction with its fragment crystallizable (Fc) region. Specifically, the Fc region spanning the interface of CH2 and CH3 domains interacts with the FcRn on the surface of cells to regulate IgG homeostasis. This interaction is favored by an acidic interaction after IgG pinocytosis and thus IgG is protected from degradation. The endocytosed IgG is then recycled back to the cell surface and released into the blood stream at a slightly alkaline pH thereby maintaining sufficient serum IgG for proper function. Accordingly, the pharmacokinetic profile of IgGs depend on the structural and functional properties of their Fc regions.

Fc regions are also responsible for antibody effector functions, such as complement-dependent cytotoxicity (CDC), antibody-dependent cellular cytotoxicity (ADCC) and antibody-dependent cellular phagocytosis (ADCP). These effector functions rely on the interactions of Fc regions with FcγRs. Therefore, engineering Fc regions to tune their interactions with FcγRs has emerged as a promising approach for enhancing the activity of therapeutic antibodies.

In human health, decades of Fe engineering have led to the identification of numerous mutations that improved pharmacokinetics and effector functions. To date, no such Fc engineering study was reported in bovine.

Accordingly, there exists a need for novel bovine IgG Fc region mutations to improve various characteristics of bovine IgGs.

### SUMMARY OF THE INVENTION

The invention relates to mutant bovine IgGs that exhibit desired characteristics, relative to wild-type bovine IgGs. Specifically, the inventors of the instant application have found that substituting an amino acid residue at position 216, 234, 235, 237, 270, 329, 330, 331, 432, 434, 437, or 433 (numbered according to the Eu index as in Kabat) with another amino acid surprisingly and unexpectedly exhibited a desired effect. In an exemplary embodiment, the undexpected desired effects include, but not limited to, enhanced affinity to FcRn; reduced complement-dependent cytotoxicity (CDC); reduced antibody-dependent cellular cytotoxicity (ADCC); reduced antibody-dependent cellular phagocytosis (ADCP); reduced binding to Fc gamma receptor (bFcgR); or a combination thereof.

In one aspect, the invention provides a modified IgG comprising: a bovine IgG constant domain comprising at least one amino acid substitution relative to a wild-type bovine IgG constant domain, wherein said substitution is at amino acid residue 216, 234, 235, 237, 270, 329, 330, 331, 432, 434, 437, or 433.

In one exemplary embodiment, the bovine IgG constant domain is an IgG1 constant domain that comprises one or more of substitutions of P329S, A330S, P331S, P234A, L235A, G237A, D216E, D270E, L432A, N434A, and T437A.

In another exemplary embodiment, the bovine IgG constant domain is an IgG2 constant domain that comprises one or more of substitutions of A330S, L432A, N434A, and M437A.

In yet another exemplary embodiment, the bovine IgG constant domain is an IgG3 constant domain that comprises one or more of substitutions of P329S, A330S, P331S, P234A, L235A, G237A, D270E, L432A, N434A, T437A, and R433H.

In another aspect, the invention provides a polypeptide comprising: a bovine IgG constant domain comprising one or more amino acid substitutions of the invention described herein.

In yet another aspect, the invention provides an antibody or a molecule comprising: a bovine IgG constant domain comprising one or more amino acid substitutions of the invention described herein.

In a further aspect, the invention provides a method for producing or manufacturing an antibody or a molecule, the method comprising: providing a vector or a host cell having a nucleic acid sequence that encodes an antibody, wherein said antibody comprises a bovine IgG constant domain comprising one or more amino acid substitutions of the invention described herein.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates target cell killing functions triggered by IgG Fc binding to Fc gamma receptors on the surface of effector cells.

FIG. 2 shows the sequence alignment bovine IgG subclasses. Three representative allotypes are shown. CH1, hinge, CH2, and CH3 domains are as follows: CH1: residues 1-98; hinge: 99 to vertical lines; CH2: vertical lines to 243; CH3: 244-351. Cysteines involved in Inter-heavy chain disulfide bonds are in bold and underlined. The 3′extension of CH1 exon in cow IgG3a is italicized. Bolded amino acids just downstream from the hinge represent the “Winter” or “LALA” sites. For bIgG1a and bIgG3a, these sites are LPGG and PLGG, respectively. This site is absent in bIgG2a. Box includes “PAP” region in bIgG1a and bIgG3a CH2 and “SAS” region in bIgG2a. Double-underlined arginine (R) in bIgG3a is residue involved in point mutation to histidine (H).

FIG. 3 shows the sequence alignment of the three unique alleles of bovine IgG1 subclass. Bold: amino acid “DP” regions mutated on bIgG1a to mitigate protease clipping. Underlined: amino acids mutated on bIgG1a to knock-out effector function. Because bIgG1d has synonymous amino acid sequence as bIgG1b, it is not included in the alignment of bIgG1 allotypes. All mutations made to bIgG1 are identical for the a and b allotypes.

FIG. 4 shows the sequence alignment of two reported alleles of the bovine IgG2 subclass, bIgG2a (NCBI X16702.1) and bIgG2b (NCBI S82407) with the sequence of an IgG2 antibody isolated from cow. Bold: amino acids mutated on bIgG2a to knock-out effector function. Inventors have isolated an IgG antibody from a dairy cow and sequencing revealed a 2-residue deviation from that reported for bIgG2a, as shown in FIG. 4 as “bIgG2a from cow seq”. This sequence may represent a new allotype for bIgG2, but for the purposes of this patent application, it will be referred to as “bIgG2a” due to near-identical alignment with bIgG2a. All mutations made to bIgG2 are identical for the a and b allotypes.

FIG. 5 shows the sequence alignment of the two alleles of the bovine IgG3 subclass. Bold: amino acids mutated on bIgG3a to knock-out effector function. Underlined: the residue mutated on bIgG3a to enhance the affinity to bFcRn. All mutations made to bIgG3 are identical for the a and b allotypes.

FIG. 6 shows bovine IgG1a and bIgG1a Fc mutants for knock out of effector function. Bold: the residues involved in the Winter mutation L234A_P235A_G237A*. Box: the residues involved in the PAP-to-SAS (P329S_P331S*), PAP-to-SAP (P329S*) mutations, and the PAP-to-PSS (SS mutation; A330S_P331S*) mutation that is combined with the Winter mutation. Vertical line: start of the Fc region in the CTLA4-Fc fusion proteins. *The amino acid residues of the mutations are numbered according to the Eu index as in Kabat.

FIGS. 7A and 7B show cell-based complement-dependent cytotoxicity activity of bovine IgG1a, IgG1b and variants of both allotypes.

FIG. 8 shows antibody homology modeling of bIgG1a_WINSAS, bIgG1b_WINSAS, bIgG1c_WINSAS and bIgG1d_WINSAS. A) Overlay of the protein models with a zoomed-in sub-panel showing WINSAS residues (arrows). B) Root Mean Square Deviation (RMSD) comparisons (in angstroms) of WINSAS mutants of bIgG1a_WINSAS, bIgG1b_WINSAS, bIgG1c_WINSAS and bG1d_WINSAS allotypes.

FIG. 9 shows Fc mutations on bovine IgG1a subclass for the elimination of Antibody Dependent Cellular Phagocytosis in a cell-based assay.

FIG. 10 shows analytical SEC of wild-type bovine IgG1a molecule.

FIG. 11A shows mass spectrometric analysis of Bovine IgG1a wild type (WT) Fc. FIG. 11B shows mass spectrometric analysis of Bovine IgG1a double mutation DP Site 1 & 2.

FIG. 12 shows bovine IgG1a and bIgG1a DP-to-EP Fc mutants for eliminating cleavage site. Bold: “DP1” (site 1) mutation to EP; Underlined: “DP2” (site 2) mutation to EP.

FIG. 13 shows antibody homology modeling of bIgG1a_DP1_DP2, bIgG1b_DP1_DP2, bIgG1c_DP1_DP2 and bIgG1d_DP1_DP2. A) Overlay of the protein models with a zoomed-in sub-panel showing DP1 and DP2 residues (arrows). B) Root Mean Square Deviation (RMSD) comparisons of DP1_DP2 mutants of bovine G1a, G1b, G1c and G1d allotypes.

FIG. 14 shows bovine IgG2a and bIgG2a Fc mutants for knock out of effector function. Bold in first line: the three residues L432*, N434*, and M437* mutated to alanine as independent mutations or in combinations. Vertical line: start of the Fc region in the CTLA4-Fc fusion proteins. *The amino acid residues of the mutations are numbered according to the Eu index as in Kabat.

FIGS. 15A and 15B show that Fc mutations on bovine IgG2a and IgG2b subclass eliminate cell-based complement-dependent cytotoxicity activity.

FIG. 16 shows antibody homology modeling of bIgG2a_L432A_N434A_M437A and bIgG2b_L432A_N434A_M437A. A) Overlay of the protein models with a zoomed-in sub-panel showing L432A_N434A_M437A residues (arrows). B) Root Mean Square Deviation (RMSD) comparisons of L432A_N434A_M437A mutants of bovine IgG2a and IgG2b allotypes.

FIG. 17 shows that Fc mutations on bovine IgG2a subclass eliminate Antibody Dependent Cellular Phagocytosis in a cell-based assay.

FIG. 18 shows bovine IgG3a and bIgG3a Fc mutants for knock out of effector function. Bold: the residues involved in the Winter mutation (P234A_L235A_G237A*). Box: the residues involved in the PAP-to-SAS (P329S_P331S*) and PAP-to-SAP (P329S*) mutations. Vertical line: start of the Fc region in the CTLA4-Fc fusion proteins. *The amino acid residues of the mutations are numbered according to the Eu index as in Kabat.

FIGS. 19A and 19B show Fc mutations on bovine IgG3a and IgG3b subclass for the elimination of complement-dependent cytotoxicity in a cell-based assay.

FIG. 20 shows antibody homology modeling of bIgG3a_WINSAS and bIgG3b_WINSAS. A) Overlay of the protein models with a zoomed-in sub-panel showing WINSAS residues (arrows). B) Root Mean Square Deviation (RMSD) comparisons of WINSAS mutants of bovine IgG3a and IgG3b allotypes.

FIG. 21 shows that Fc mutations on bovine IgG3a subclass eliminate Antibody Dependent Cellular Phagocytosis in a cell-based assay.

FIG. 22 shows bovine IgG3a and Fc mutant for improved bFcRn binding. Bold arginine (R) is residue involved in point mutation to histidine (H), R433H, numbered according to the Eu index as in Kabat.

FIG. 23 shows the alignment of the amino acid sequences of human IgG1, bovine IgG1a, bovine IgG2a, and bovine IgG3a. The amino acid residues are numbered according to the Eu index as in Kabat. Amino acid residues for the other alleles for IgG1 (b, c, d), IgG2 (b), and IgG3 (b) were also numbered according to the Eu index shown in FIG. 23.

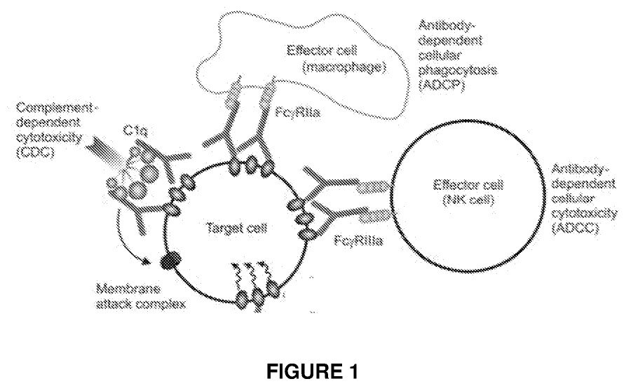

### BRIEF DESCRIPTION OF THE SEQUENCE LISTINGS

SEQ ID NO.: 1 refers to the amino acid sequence of IgG1a Wildtype (NCBI ID number 1S82409).

SEQ ID NO.: 2 refers to the amino acid sequence of IgG1b Wildtype (NCBI ID number X16701).

SEQ ID NO.: 3 refers to the amino acid sequence of IgG1c Wildtype (NCBI ID number DQ452014.1).

SEQ ID NO.: 4 refers to the amino acid sequence of IgG1d Wildtype (NCBI ID number X62916.1).

SEQ ID NO.: 5 refers to the amino acid sequence of IgG2a Wildtype (NCBI ID number X16702.1).

SEQ ID NO.: 6 refers to the amino acid sequence of IgG2b Wildtype (NCBI ID number S82407).

SEQ ID NO.: 7 refers to the amino acid sequence of IgG3a Wildtype.

SEQ ID NO.: 8 refers to the amino acid sequence of IgG3b Wildtype.

SEQ ID NO.: 9 refers to the amino acid sequence of IgG2a Wildtype from Dairy Cow.

SEQ ID NO.: 10 refers to the amino acid sequence of IgG1a Having DP1 Mutation.

SEQ ID NO.: 11 refers to the amino acid sequence of IgG1a Having DP2 Mutation.

SEQ ID NO.: 12 refers to the amino acid sequence of IgG1a Having DP1 and DP2 Mutations.

SEQ ID NO.: 13 refers to the amino acid sequence of IgG1a Wildtype Fragment Physical Positions 99-329.

SEQ ID NO.: 14 refers to the amino acid sequence of IgG1a Fragment Having SAP Mutation.

SEQ ID NO.: 15 refers to the amino acid sequence of IgG1a Fragment Having SAS Mutation.

SEQ ID NO.: 16 refers to the amino acid sequence of IgG1a Fragment Having Winter Mutations.

SEQ ID NO.: 17 refers to the amino acid sequence of IgG1a Fragment Having Winter and SAS Mutations.

SEQ ID NO.: 18 refers to the amino acid sequence of IgG1a Fragment Having Winter and SS Mutations.

SEQ ID NO.: 19 refers to the amino acid sequence of IgG2a Having Mutation L432A.

SEQ ID NO.: 20 refers to the amino acid sequence of IgG2a Having Mutation N434A.

SEQ ID NO.: 21 refers to the amino acid sequence of IgG2a Having Mutation M437A.

SEQ ID NO.: 22 refers to the amino acid sequence of IgG2a Having Mutations L432A and M437A.

SEQ ID NO.: 23 refers to the amino acid sequence of IgG2a Having Mutations N434A and M437A.

SEQ ID NO.: 24 refers to the amino acid sequence of IgG2a Having Mutations L432A, N434A, and M437A.

SEQ ID NO.: 25 refers to the amino acid sequence of IgG2a Having Mutations L432A and N434A.

SEQ ID NO.: 26 refers to the amino acid sequence of IgG3a Wildtype Fragment Physical Positions 99-352.

SEQ ID NO.: 27 refers to the amino acid sequence of IgG3a Fragment Having SAP Mutation.

SEQ ID NO.: 28 refers to the amino acid sequence of IgG3a Fragment Having SAS Mutation.

SEQ ID NO.: 29 refers to the amino acid sequence of IgG3a Fragment Having Winter Mutation.

SEQ ID NO.: 30 refers to the amino acid sequence of IgG3a Fragment Having Winter and SAS Mutations.

SEQ ID NO.: 31 refers to the amino acid sequence of IgG3a Having Mutation R433H.

SEQ ID NO.: 32 refers to the amino acid sequence of human IgG1.

SEQ ID NO.: 33 refers to the amino acid sequence of IgG1b Wildtype.

SEQ ID NO.: 34 refers to the nucleic acid sequence of IgG1b Wildtype.

SEQ ID NO.: 35 refers to the amino acid sequence of IgG1b having Winter mutation.

SEQ ID NO.: 36 refers to the nucleic acid sequence of IgG1b having Winter mutation.

SEQ ID NO.: 37 refers to the amino acid sequence of IgG1b having WinSS mutation.

SEQ ID NO.: 38 refers to the nucleic acid sequence of IgG1b having WinSS mutation.

SEQ ID NO.: 39 refers to the amino acid sequence of IgG1b having WinSAS mutation.

SEQ ID NO.: 40 refers to the nucleic acid sequence of IgG1b having WinSAS mutation.

SEQ ID NO.: 41 refers to the amino acid sequence of IgG1b having SAS mutation.

SEQ ID NO.: 42 refers to the nucleic acid sequence of IgG1b having SAS mutation.

SEQ ID NO.: 43 refers to the amino acid sequence of IgG1b having SAP mutation.

SEQ ID NO.: 44 refers to the nucleic acid sequence of IgG1b having SAP mutation.

SEQ ID NO.: 45 refers to the amino acid sequence of IgG1b having D216E mutation.

SEQ ID NO.: 46 refers to the nucleic acid sequence of IgG1b having D216E mutation.

SEQ ID NO.: 47 refers to the amino acid sequence of IgG1b having D270E mutation.

SEQ ID NO.: 48 refers to the nucleic acid sequence of IgG1b having D270E mutation.

SEQ ID NO.: 49 refers to the amino acid sequence of IgG1b having D216E and D270E mutations.

SEQ ID NO.: 50 refers to the nucleic acid sequence of IgG1b having D216E and D270E mutations.

SEQ ID NO.: 51 refers to the amino acid sequence of IgG2b Wildtype.

SEQ ID NO.: 52 refers to the nucleic acid sequence of IgG2b Wildtype.

SEQ ID NO.: 53 refers to the amino acid sequence of IgG2b having L432A mutation.

SEQ ID NO.: 54 refers to the nucleic acid sequence of IgG2b having L432A mutation.

SEQ ID NO.: 55 refers to the amino acid sequence of IgG2b having N434A mutation.

SEQ ID NO.: 56 refers to the nucleic acid sequence of IgG2b having N434A mutation.

SEQ ID NO.: 57 refers to the amino acid sequence of IgG2b having M437A mutation.

SEQ ID NO.: 58 refers to the nucleic acid sequence of IgG2b having M437A mutation.

SEQ ID NO.: 59 refers to the amino acid sequence of IgG2b having L432A and N434A mutations.

SEQ ID NO.: 60 refers to the nucleic acid sequence of IgG2b having L432A and N434A mutations.

SEQ ID NO.: 61 refers to the amino acid sequence of IgG2b having L432A and M437A mutations.

SEQ ID NO.: 62 refers to the nucleic acid sequence of IgG2b having L432A and M437A mutations.

SEQ ID NO.: 63 refers to the amino acid sequence of IgG2b having N434A and M437A mutations.

SEQ ID NO.: 64 refers to the nucleic acid sequence of IgG2b having N434A and M437A mutations.

SEQ ID NO.: 65 refers to the amino acid sequence of IgG2b having L432A, N434A and M437A mutations.

SEQ ID NO.: 66 refers to the nucleic acid sequence of IgG2b having L432A, N434A and M437A mutations.

SEQ ID NO.: 67 refers to the amino acid sequence of IgG3b Wildtype.

SEQ ID NO.: 68 refers to the nucleic acid sequence of IgG3b Wildtype.

SEQ ID NO.: 69 refers to the amino acid sequence of IgG3b having Winter mutation.

SEQ ID NO.: 70 refers to the nucleic acid sequence of IgG3b having Winter mutation.

SEQ ID NO.: 71 refers to the amino acid sequence of IgG3b having WinSAS mutation.

SEQ ID NO.: 72 refers to the nucleic acid sequence of IgG3b having WinSAS mutation.

SEQ ID NO.: 73 refers to the amino acid sequence of IgG3b having SAS mutation.

SEQ ID NO.: 74 refers to the nucleic acid sequence of IgG3b having SAS mutation.

SEQ ID NO.: 75 refers to the amino acid sequence of IgG3b having SAP mutation.

SEQ ID NO.: 76 refers to the nucleic acid sequence of IgG3b having SAP mutation.

SEQ ID NO.: 77 refers to the amino acid sequence of IgG3b having R433H mutation.

SEQ ID NO.: 78 refers to the nucleic acid sequence of IgG3b having R433H mutation.

SEQ ID NO.: 79 refers to the flanking amino acid sequence of bIgG1bWin (L234A_P235A_G237A): LPGG to AAGA.

SEQ ID NO.: 80 refers to the flanking nucleic acid sequence of bIgG1bWin (L234A_P235A_G237A): LPGG to AAGA.

SEQ ID NO.: 81 refers to the flanking amino acid sequence of bIgG1bWinSS (A330S_P331S): PAP to PSS.

SEQ ID NO.: 82 refers to the flanking nucleic acid sequence of bIgG1bWinSS (A330S_P331S): PAP to PSS.

SEQ ID NO.: 83 refers to the flanking amino acid sequence of bIgG1bSAS (P329S_P331S): PAP to SAS.

SEQ ID NO.: 84 refers to the flanking nucleic acid sequence of bIgG1bSAS (P329S_P331S): PAP to SAS.

SEQ ID NO.: 85 refers to the flanking amino acid sequence of bIgG1bSAP (P329S): PAP to SAP.

SEQ ID NO.: 86 refers to the flanking nucleic acid sequence of bIgG1bSAP (P329S): PAP to SAP.

SEQ ID NO.: 87 refers to the flanking amino acid sequence of bIgG1b_DP1 (D216E): DP1 to EP1.

SEQ ID NO.: 88 refers to the flanking nucleic acid sequence of bIgG1b_DP1 (D216E): DP1 to EP1.

SEQ ID NO.: 89 refers to the flanking amino acid sequence of bIgG1b_DP2 (D270E): DP2 to EP2.

SEQ ID NO.: 90 refers to the flanking nucleic acid sequence of bIgG1b_DP2 (D270E): DP2 to EP2.

SEQ ID NO.: 91 refers to the flanking amino acid sequence of bIgG2b_L432A: LHNHYM to AHNHYM.

SEQ ID NO.: 92 refers to the flanking nucleic acid sequence of bIgG2b_L432A: LHNHYM to AHNHYM.

SEQ ID NO.: 93 refers to the flanking amino acid sequence of bIgG2b_N434A: LHNHYM to LHAHYM.

SEQ ID NO.: 94 refers to the flanking nucleic acid sequence of bIgG2b_N434A: LHNHYM to LHAHYM.

SEQ ID NO.: 95 refers to the flanking amino acid sequence of bIgG2b_M437A: LHNHYM to LHNHYA.

SEQ ID NO.: 96 refers to the flanking nucleic acid sequence of bIgG2b_M437A: LHNHYM to LHNHYA.

SEQ ID NO.: 97 refers to the flanking amino acid sequence of bIgG2b_L432A_N434A: LHNHYM to AHNHYM; LHNHYM to LHAHYM.

SEQ ID NO.: 98 refers to the flanking nucleic acid sequence of bIgG2b_L432A_N434A: LHNHYM to AHNHYM; LHNHYM to LHAHYM.

SEQ ID NO.: 99 refers to the flanking amino acid sequence of bIgG2b_L432A_M437A: LHNHYM to AHNHYM; LHNHYM to LHNHYA.

SEQ ID NO.: 100 refers to the flanking nucleic acid sequence of bIgG2b_L432A_M437A: LHNHYM to AHNHYM; LHNHYM to LHNHYA.

SEQ ID NO.: 101 refers to the flanking amino acid sequence of bIgG2b_N434A_M437A: LHNHYM to LHAHYM; LHNHYM to LHNHYA.

SEQ ID NO.: 102 refers to the flanking nucleic acid sequence of bIgG2b_N434A_M437A: LHNHYM to LHAHYM; LHNHYM to LHNHYA.

SEQ ID NO.: 103 refers to the flanking amino acid sequence of bIgG2b_L432A_N434A_M437A: LHNHYM to AHNHYM; LHNHYM to LHAHYM; LHNHYM to LHNHYA.

SEQ ID NO.: 104 refers to the flanking nucleic acid sequence of bIgG2b_L432A_N434A_M437A: LHNHYM to AHNHYM; LHNHYM to LHAHYM; LHNHYM to LHNHYA.

SEQ ID NO.: 105 refers to the flanking amino acid sequence of bIgG3bWin (P234A_L235A_G237A): PLGG to AAGA.

SEQ ID NO.: 106 refers to the flanking nucleic acid sequence of bIgG3bWin (P234A_L235A_G237A): PLGG to AAGA.

SEQ ID NO.: 107 refers to the flanking amino acid sequence of bIgG3bSAS (P329S_P331S): PAP to SAS.

SEQ ID NO.: 108 refers to the flanking nucleic acid sequence of bIgG3bSAS (P329S_P331S): PAP to SAS.

SEQ ID NO.: 109 refers to the flanking amino acid sequence of bIgG3bSAP (P329S): PAP to SAP.

SEQ ID NO.: 110 refers to the flanking nucleic acid sequence of bIgG3bSAP (P329S): PAP to SAP.

SEQ ID NO.: 111 refers to the flanking amino acid sequence of bIgG3b_R433H: ALRNH to ALHNH.

SEQ ID NO.: 112 refers to the flanking nucleic acid sequence of bIgG3b_R433H: ALRNH to ALHNH.

SEQ ID NO.:113 refers to the amino acid sequence of Biotin Acceptor Peptide (BAP).

### DETAILED DESCRIPTION OF THE INVENTION

The present subject matter may be understood more readily by reference to the following detailed description which forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

### Definitions

In the present disclosure the singular forms “a,” “an,” and “the” include the plural reference, and reference to a particular numerical value includes at least that particular value, unless the context clearly indicates otherwise. Thus, for example, a reference to “a molecule” or “a compound” is a reference to one or more of such molecules or compounds and equivalents thereof known to those skilled in the art, and so forth. The term “plurality”, as used herein, means more than one. When a range of values is expressed, another embodiment incudes from the one particular and/or to the other particular value. Similarly, when values are expressed as approximations, by use of the antecedent “about,” it is understood that the particular value forms another embodiment. All ranges are inclusive and combinable.

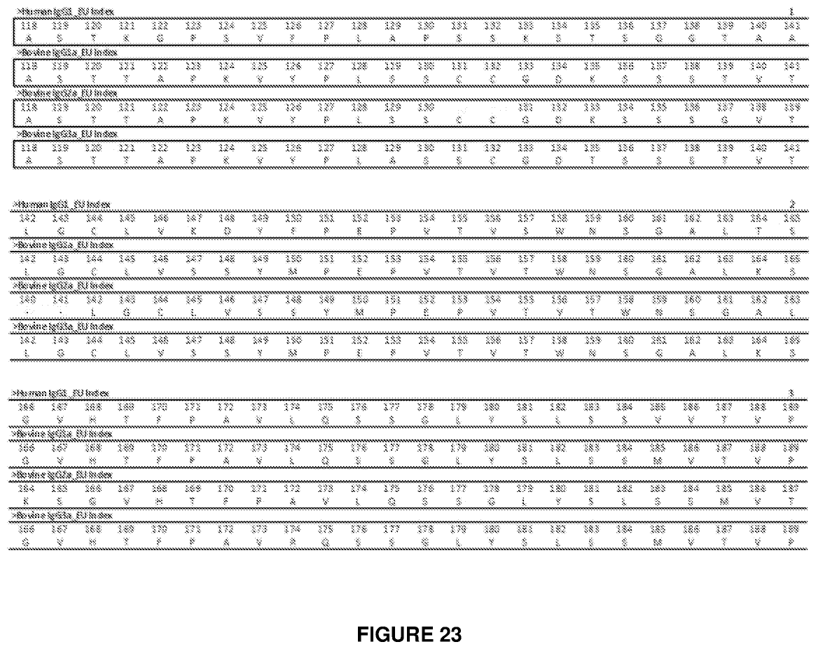

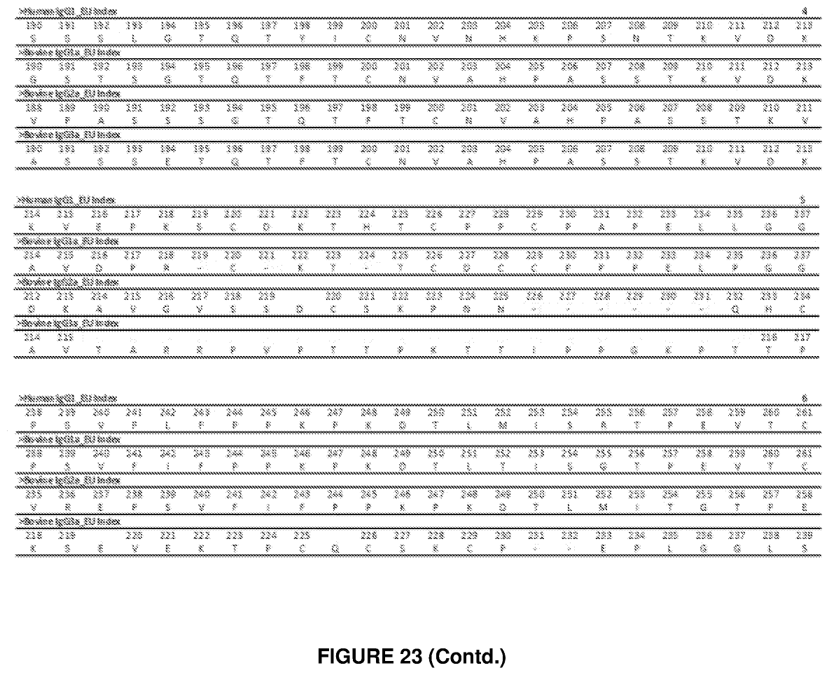

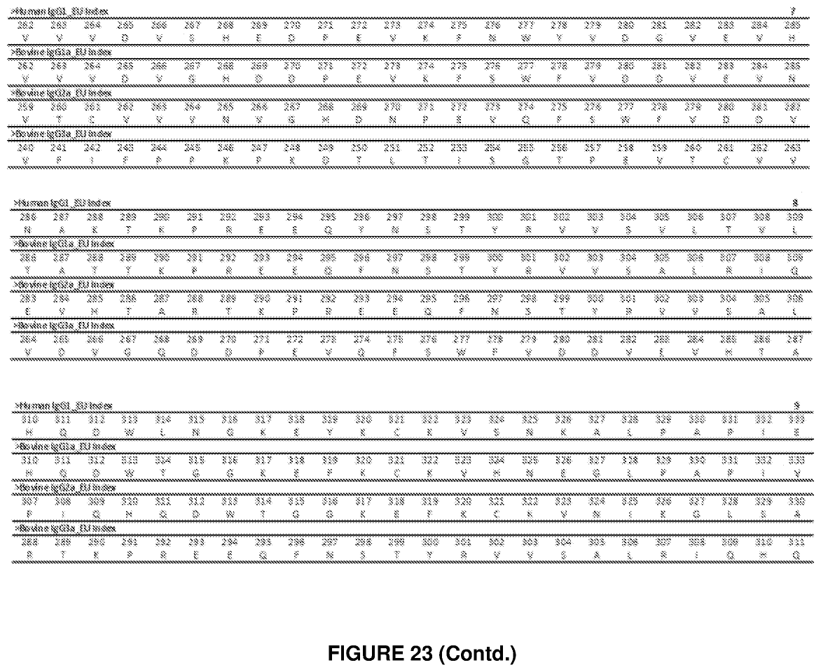

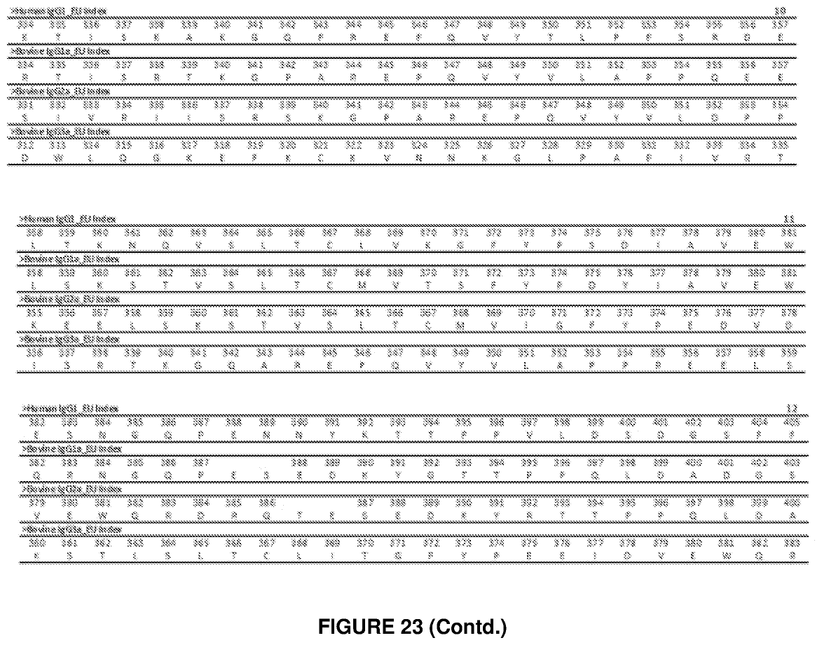

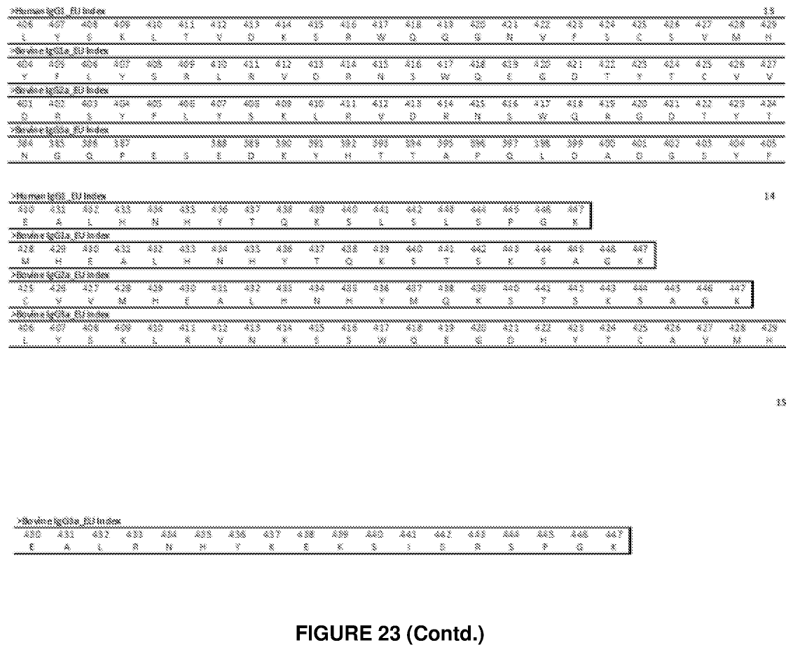

In the specification and claims, the numbering of the amino acid residues in an immunoglobulin heavy chain is that of the Eu index as in Kabat, Sequences of Proteins of Immunological Interest, 5th Ed. Public Health Service, National Institutes of Health, Bethesda, Md. (1991). The “Eu index as in Kabat” refers to the residue numbering of the IgG antibody and is reflected herein in FIG. 23.

The term “isolated” when used in relation to a nucleic acid is a nucleic acid that is identified and separated from at least one contaminant nucleic acid with which it is ordinarily associated in its natural source. Isolated nucleic acid is in a form or setting different from that in which it is found in nature. Isolated nucleic acid molecules therefore are distinguished from the nucleic acid molecule as it exists in natural cells. An isolated nucleic acid molecule includes a nucleic acid molecule contained in cells that ordinarily express the polypeptide encoded therein where, for example, the nucleic acid molecule is in a plasmid or a chromosomal location different from that of natural cells. The isolated nucleic acid may be present in single-stranded or double-stranded form. When an isolated nucleic acid molecule is to be utilized to express a protein, the oligonucleotide or polynucleotide will contain at a minimum the sense or coding strand, but may contain both the sense and anti-sense strands (i.e., may be double-stranded).

A nucleic acid molecule is “operably linked” or “operably attached” when it is placed into a functional relationship with another nucleic acid molecule. For example, a promoter or enhancer is operably linked to a coding sequence of nucleic acid if it affects the transcription of the sequence; or a ribosome binding site is operably linked to a coding sequence of nucleic acid if it is positioned so as to facilitate translation. A nucleic acid molecule encoding a variant Fc region is operably linked to a nucleic acid molecule encoding a heterologous protein (i.e., a protein or functional fragment thereof which does not, as it exists in nature, comprise an Fc region) if it is positioned such that the expressed fusion protein comprises the heterologous protein or functional fragment thereof adjoined either upstream or downstream to the variant Fc region polypeptide; the heterologous protein may by immediately adjacent to the variant Fc region polypeptide or may be separated therefrom by a linker sequence of any length and composition. Likewise, a polypeptide (used synonymously herein with “protein”) molecule is “operably linked” or “operably attached” when it is placed into a functional relationship with another polypeptide.

As used herein the term “functional fragment” when in reference to a polypeptide or protein (e.g., a variant Fc region, or a monoclonal antibody) refers to fragments of that protein which retain at least one function of the full-length polypeptide. The fragments may range in size from six amino acids to the entire amino acid sequence of the full-length polypeptide minus one amino acid. A functional fragment of a variant Fc region polypeptide of the present invention retains at least one “amino acid substitution” as herein defined. A functional fragment of a variant Fc region polypeptide retains at least one function known in the art to be associated with the Fc region (e.g., ADCC, CDC, Fc receptor binding, C1q binding, down regulation of cell surface receptors or may, e.g., increase the in vivo or in vitro half-life of a polypeptide to which it is operably attached).

The term “purified” or “purify” refers to the substantial removal of at least one contaminant from a sample. For example, an antigen-specific antibody may be purified by complete or substantial removal (at least 90%, 91%, 92%, 93%, 94%, 95%, or more preferably at least 96%, 97%, 98% or 99%) of at least one contaminating non-immunoglobulin protein; it may also be purified by the removal of immunoglobulin protein that does not bind to the same antigen. The removal of non-immunoglobulin proteins and/or the removal of immunoglobulins that do not bind a particular antigen results in an increase in the percent of antigen-specific immunoglobulins in the sample. In another example, a polypeptide (e.g., an immunoglobulin) expressed in bacterial host cells is purified by the complete or substantial removal of host cell proteins; the percent of the polypeptide is thereby increased in the sample.

The term “native” as it refers to a polypeptide (e.g., Fc region) is used herein to indicate that the polypeptide has an amino acid sequence consisting of the amino acid sequence of the polypeptide as it commonly occurs in nature or a naturally occurring polymorphism thereof. A native polypeptide (e.g., native Fc region) may be produced by recombinant means or may be isolated from a naturally occurring source.

The term “expression vector” as used herein refers to a recombinant DNA molecule containing a desired coding sequence and appropriate nucleic acid sequences necessary for the expression of the operably linked coding sequence in a particular host organism.

As used herein, the term “host cell” refers to any eukaryotic or prokaryotic cell (e.g., bacterial cells such as E. coli, CHO cells, yeast cells, mammalian cells, avian cells, amphibian cells, plant cells, fish cells, and insect cells), whether located in vitro or in situ, or in vivo

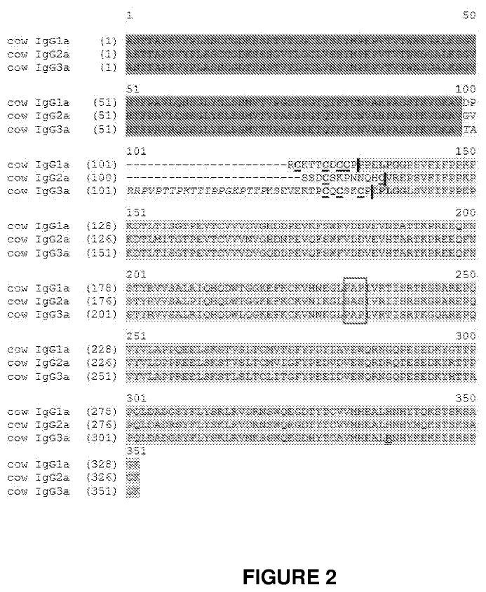

As used herein, the term “Fc region” refers to a C-terminal region of an immunoglobulin heavy chain. The “Fc region” may be a native sequence Fc region or a variant Fc region. Although the generally accepted boundaries of the Fc region of an immunoglobulin heavy chain might vary, the bovine IgG heavy chain Fc region is usually defined to stretch, for example, from the vertical lines to the c-terminus in FIG. 2. In some embodiments, variants comprise only portions of the Fc region and can include or not include the carboxy-terminus. The Fc region of an immunoglobulin generally comprises two constant domains, CH2 and CH3. In some embodiments, variants having one or more of the constant domains are contemplated. In other embodiments, variants without such constant domains (or with only portions of such constant domains) are contemplated.

The “CH2 domain” of a bovine IgG Fc region refers to, for example, the residues starting at the vertical lines and extending to residue 243 in FIG. 2. The CH2 domain is unique in that it is not closely paired with another domain. Two N-linked branched carbohydrate chains are interposed between the two CH2 domains of an intact native IgG molecule.

The “CH3 domain” of a bovine IgG Fc region generally is the stretch of residues C-terminal to a CH2 domain in an Fc region, for example, residues 244 to the c-terminus in FIG. 2.

A “functional Fc region” possesses an “effector function” of a native sequence Fc region. Examples of effector functions include, but are not limited to: C1q binding; complement dependent cytotoxicity (CDC); Fc receptor binding; antibody-dependent cell-mediated cytotoxicity (ADCC); antibody-dependent cellular phagocytosis (ADCP); down regulation of cell surface receptors (e.g., B cell receptor; BCR), etc. Such effector functions may require the Fc region to be operably linked to a binding domain (e.g., an antibody variable domain) and can be assessed using various assays (e.g., Fc binding assay, ADCC assays, CDC assays, ADCP assays, target cell depletion from whole or fractionated blood samples, etc.).

A “native sequence Fc region” or “wild type Fc region” refers to an amino acid sequence that is identical to the amino acid sequence of an Fc region commonly found in nature. Exemplary native sequence bovine Fc regions are from the vertical lines to the c-terminus in FIG. 2.

A “variant Fc region” comprises an amino acid sequence that differs from that of a native sequence Fc region (or fragment thereof) by virtue of at least one “amino acid substitution” as defined herein. In preferred embodiments, the variant Fc region has at least one amino acid substitution compared to a native sequence Fc region or in the Fc region of a parent polypeptide, preferably 1, 2, 3, 4 or 5 amino acid substitutions in a native sequence Fc region or in the Fc region of the parent polypeptide. In an alternative embodiment, a variant Fc region may be generated according to the methods herein disclosed and this variant Fc region can be fused to a heterologous polypeptide of choice, such as an antibody variable domain or a non-antibody polypeptide, e.g., binding domain of a receptor or ligand.

As used herein, the term “derivative” in the context of polypeptides refers to a polypeptide that comprises and amino acid sequence which has been altered by introduction of an amino acid residue substitution. The term “derivative” as used herein also refers to a polypeptide which has been modified by the covalent attachment of any type of molecule to the polypeptide. For example, but not by way of limitation, an antibody may be modified, e.g., by glycosylation, acetylation, pegylation, phosphorylation, amidation, derivatization by known protecting/blocking groups, proteolytic cleavage, linkage to a cellular ligand or other protein, etc. A derivative polypeptide may be produced by chemical modifications using techniques known to those of skill in the art, including, but not limited to specific chemical cleavage, acetylation, formylation, metabolic synthesis of tunicamycin, etc. Further, a derivative polypeptide possesses a similar or identical function as the polypeptide from which it was derived. It is understood that a polypeptide comprising a variant Fc region of the present invention may be a derivative as defined herein, preferably the derivatization occurs within the Fc region.

“Substantially of bovine origin” as used herein in reference to a polypeptide (e.g., an Fc region or a monoclonal antibody), indicates the polypeptide has an amino acid sequence at least 80%, at least 85%, more preferably at least 90%, 91%, 92%, 93%, 94% or even more preferably at least 95%, 96%, 97%, 98% or 99% homologous to that of a native bovine amino polypeptide.

The terms “Fc receptor” or “FcR” are used to describe a receptor that binds to an Fc region (e.g., the Fc region of an antibody). The preferred FcR is a native sequence FcR. Moreover, a preferred FcR is one which binds an IgG antibody Fc region, an Fc gamma receptor or “FcgR”, and includes receptors of the Fc gamma RI (FcgR1), Fc gamma RII (FcgR2), Fc gamma RIII (FcgR3) subclasses, including allelic variants and alternatively spliced forms of these receptors as well as the novel bovine Fc gamma 2R (bFcg2R or bFcg2R). Another preferred FcR includes the neonatal receptor, FcRn, which is responsible for the transfer of maternal IgGs to the fetus (Guyer et al., J. Immunol. 117:587 (1976) and Kim et al., J. Immunol. 24:249 (1994)). Other FcRs, including those to be identified in the future, are encompassed by the term “FcR” herein.

The phrase “antibody-dependent cell-mediated cytotoxicity” and “ADCC” refer to a cell-mediated reaction in which nonspecific cytotoxic cells (e.g., nonspecific) that express FcgRs (e.g., Natural Killer (“NK”) cells, neutrophils, and macrophages) recognize bound antibody on a target cell and subsequently cause lysis of the target cells. The primary cells for mediating ADCC in humans, NK cells, express FcgR3 only, whereas monocytes express FcgR1, FcgR2 and FcgR3. Literature reports that bovine monocytes and macrophages express FcgRs for IgG1 and IgG2 isotypes, whereas neutrophils express high numbers of receptors for IgG2, Fcg2R but few or none for bIgG1.

The phrases “antibody-dependent cell-mediated phagocytosis” and “ADCP” refer to a cell-mediated reaction in which phagocytic cells (e.g., macrophages, monocytes, dendritic cells) that express FcgRs (e.g., FcgR1, FcgR2a and FcgR3) recognize bound IgG antibody Fc region on a target cell and subsequently trigger a signaling cascade leading to the engulfment of the IgG-opsonized particle (e.g., bacteria, dead tissue cells).

As used herein, the phrase “effector cells” refers to leukocytes (preferably bovine) which express one or more FcRs and perform effector functions. Preferably, the cells express at least FcgR3 and perform ADCC effector function. Examples of leukocytes which mediate ADCC include PBMC, NK cells, monocytes, macrophage, cytotoxic T cells and neutrophils. The effector cells may be isolated from a native source (e.g., from blood or PBMCs). In one example, the leukocytes express FcgR1, or other relevant Fc gamma receptor, and trigger ADCP function.

A variant polypeptide with “altered” Fc receptor binding affinity is one which has either enhanced (i.e., increased, greater or higher) or diminished (i.e., reduced, decreased or lesser) Fc receptor binding affinity compared to the variant's parent polypeptide or to a polypeptide comprising a native Fc. A variant polypeptide which displays increased binding or increased binding affinity to an Fc receptor binds Fc receptor with greater affinity than the parent polypeptide. A variant polypeptide which displays decreased binding or decreased binding affinity to an Fc receptor, binds Fc receptor with lower affinity than its parent polypeptide. Such variants which display decreased binding to an Fc receptor may possess little or no appreciable binding to an Fc receptor, e.g., 0-20% binding to Fc receptor the Fc receptor compared to a parent polypeptide. A variant polypeptide which binds an Fc receptor with “enhanced affinity” as compared to its parent polypeptide, is one which binds Fc receptor with higher binding affinity than the parent polypeptide, when the amounts of variant polypeptide and parent polypeptide in a binding assay are essentially the same, and all other conditions are identical. For example, a variant polypeptide with enhanced Fc receptor binding affinity may display from about 1.10 fold to about 100 fold (more typically from about 1.2 fold to about 50 fold) increase in Fc receptor binding affinity compared to the parent polypeptide, where Fc receptor binding affinity is determined, for example, in an ELISA assay or other method available to one of ordinary skill in the art.

As used herein, an “amino acid substitution” refers to the replacement of at least one existing amino acid residue in a given amino acid sequence with another different “replacement” amino acid residue. The replacement residue or residues may be “naturally occurring amino acid residues” (i.e., encoded by the genetic code) and selected from: alanine (Ala); arginine (Arg); asparagine (Asn); aspartic acid (Asp); cysteine (Cys); glutamine (Gln); glutamic acid (Glu); glycine (Gly); histidine (H is); isoleucine (Ile): leucine (Leu); lysine (Lys); methionine (Met); phenylalanine (Phe); proline (Pro); serine (Ser); threonine (Thr); tryptophan (Trp); tyrosine (Tyr); and valine (Val). Substitution with one or more non-naturally occurring amino acid residues is also encompassed by the definition of an amino acid substitution herein. A “non-naturally occurring amino acid residue” refers to a residue, other than those naturally occurring amino acid residues listed above, which is able to covalently bind adjacent amino acid residues (s) in a polypeptide chain. Examples of non-naturally occurring amino acid residues include norleucine, ornithine, norvaline, homoserine and other amino acid residue analogues such as those described in Ellman et al. Meth. Enzym. 202: 301-336 (1991).

The term “assay signal” refers to the output from any method of detecting protein-protein interactions, including but not limited to, absorbance measurements from colorimetric assays, fluorescent intensity, or disintegrations per minute. Assay formats could include ELISA, FACS, or other methods. A change in the “assay signal” may reflect a change in cell viability and/or a change in the kinetic off-rate, the kinetic on-rate, or both. A “higher assay signal” refers to the measured output number being larger than another number (e.g., a variant may have a higher (larger) measured number in an ELISA assay as compared to the parent polypeptide). A “lower” assay signal refers to the measured output number being smaller than another number (e.g., a variant may have a lower (smaller) measured number in an ELISA assay as compared to the parent polypeptide).

The term “binding affinity” refers to the equilibrium dissociation constant (expressed in units of concentration) associated with each Fc receptor-Fc binding interaction. The binding affinity is directly related to the ratio of the kinetic off-rate (generally reported in units of inverse time, e.g., seconds⁻¹) divided by the kinetic on-rate (generally reported in units of concentration per unit time, e.g., molar/second). In general, it is not possible to unequivocally state whether changes in equilibrium dissociation constants (K_{D} or KD) are due to differences in on-rates, off-rates or both unless each of these parameters are experimentally determined (e.g., by BIACORE or SAPIDYNE measurements).

As used herein, the term “hinge region” refers to the stretch of amino acids that links the Fab antigen binding region to the Fc region of an antibody. Hinge regions of IgG subclasses may be aligned by placing the first and last cysteine residues forming inter-heavy chain disulfide (S—S) bonds in the same positions. As shown in FIG. 2, the hinge region, for example, in bovine IgG constant region starts at residue 99 and extends to the vertical lines.

“C1q” is a polypeptide that includes a binding site for the Fc region of an immunoglobulin. C1q together with two serine proteases, C1r and C1s, forms the complex C1, the first component of the CDC pathway.

As used herein, the term “antibody” is used interchangeably with “immunoglobulin” or “Ig,” is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity or functional activity. Single chain antibodies, and chimeric, bovine, or bovinized antibodies, as well as chimeric or CDR-grafted single chain antibodies, and the like, comprising portions derived from different species, are also encompassed by the present invention and the term “antibody”. The various portions of these antibodies can be joined together chemically by conventional techniques, synthetically, or can be prepared as a contiguous protein using genetic engineering techniques. For example, nucleic acids encoding a chimeric or bovinized chain can be expressed to produce a contiguous protein. See, e.g., U.S. Pat. Nos. 4,816,567; 4,816,397; WO 86/01533; U.S. Pat. Nos. 5,225,539; and 5,585,089 and 5,698,762. See also, Newman, R. et al. BioTechnology, 10: 1455-1460, 1993, regarding primatized antibody, and Ladner et al., U.S. Pat. No. 4,946,778 and Bird, R. E. et al., Science, 242:423-426, 1988, regarding single chain antibodies. It is understood that all forms of the antibodies comprising an Fc region (or portion thereof) are encompassed herein within the term “antibody.” Furthermore, the antibody may be labeled with a detectable label, immobilized on a solid phase and/or conjugated with a heterologous compound (e.g., an enzyme or toxin) according to methods known in the art.

As used herein, the term “antibody fragments” refers to a portion of an intact antibody. Examples of antibody fragments include, but are not limited to, linear antibodies; single-chain antibody molecules; Fc or Fc′ peptides, Fab and Fab fragments, and multispecific antibodies formed from antibody fragments. The antibody fragments preferably retain at least part of the hinge and optionally the CH1 region of an IgG heavy chain. In other preferred embodiments, the antibody fragments comprise at least a portion of the CH2 region or the entire CH2 region.

As used herein, the term “functional fragment”, when used in reference to a monoclonal antibody, is intended to refer to a portion of the monoclonal antibody that still retains a functional activity. A functional activity can be, for example, antigen binding activity or specificity, receptor binding activity or specificity, effector function activity and the like. Monoclonal antibody functional fragments include, for example, individual heavy or light chains and fragments thereof, such as VL, VH and Fd; monovalent fragments, such as Fv, Fab, and Fab′; bivalent fragments such as F(ab′)2; single chain Fv (scFv); and Fc fragments. Such terms are described in, for example, Harlowe and Lane, Antibodies: A Laboratory Manual, Cold Spring Harbor Laboratory, New York (1989); Molec. Biology and Biotechnology: A Comprehensive Desk Reference (Myers, R. A. (ed.), New York: VCH Publisher, Inc.); Huston et al., Cell Biophysics, 22:189-224 (1993); Pluckthun and Skerra, Meth. Enzymol., 178:497-515 (1989) and in Day, E. D., Advanced Immunochemistry, Second Ed., Wiley-Liss, Inc., New York, N.Y. (1990). The term functional fragment is intended to include, for example, fragments produced by protease digestion or reduction of a monoclonal antibody and by recombinant DNA methods known to those skilled in the art.

As used herein, the term “fragment” refers to a polypeptide comprising an amino acid sequence of at least 5, 15, 20, 25, 40, 50, 70, 90, 100 or more contiguous amino acid residues of the amino acid sequence of another polypeptide. In a preferred embodiment, a fragment of a polypeptide retains at least one function of the full-length polypeptide.

As used herein, the term “chimeric antibody” includes monovalent, divalent or polyvalent immunoglobulins. A monovalent chimeric antibody is a dimer formed by a chimeric heavy chain associated through disulfide bridges with a chimeric light chain. A divalent chimeric antibody is a tetramer formed by two heavy chain-light chain dimers associated through at least one disulfide bridge. A chimeric heavy chain of an antibody for use in bovine comprises an antigen-binding region derived from the heavy chain of a non-bovine antibody, which is linked to at least a portion of a bovine heavy chain constant region, such as CH1 or CH2. A chimeric light chain of an antibody for use in bovine comprises an antigen binding region derived from the light chain of a non-bovine antibody, linked to at least a portion of a bovine light chain constant region (CL). Antibodies, fragments or derivatives having chimeric heavy chains and light chains of the same or different variable region binding specificity, can also be prepared by appropriate association of the individual polypeptide chains, according to known method steps. With this approach, hosts expressing chimeric heavy chains are separately cultured from hosts expressing chimeric light chains, and the immunoglobulin chains are separately recovered and then associated. Alternatively, the hosts can be co-cultured and the chains allowed to associate spontaneously in the culture medium, followed by recovery of the assembled immunoglobulin or fragment or both the heavy and light chains can be expressed in the same host cell. Methods for producing chimeric antibodies are well known in the art (see, e.g., U.S. Pat. Nos. 6,284,471; 5,807,715; 4,816,567; and 4,816,397).

As used herein, “bovinized” forms of non-bovine (e.g., murine) antibodies (i.e., bovinized antibodies) are antibodies that contain minimal sequence, or no sequence, derived from non-bovine immunoglobulin. For the most part, bovinized antibodies are bovine immunoglobulins (recipient antibody) in which residues from a hypervariable region of the recipient are replaced by residues from a hypervariable region of a non-bovine species (donor antibody) such as mouse, rat, rabbit, human or nonhuman primate having the desired specificity, affinity, and capacity. In some instances, framework region (FR) residues of the bovine immunoglobulin are replaced by corresponding non-bovine residues. Furthermore, bovinized antibodies may comprise residues that are not found in the recipient antibody or in the donor antibody. These modifications are generally made to further refine antibody performance. In general, the bovinized antibody will comprise substantially all of at least one, and typically two, variable domains, in which all or substantially all of the hypervariable loops (CDRs) correspond to those of a non-bovine immunoglobulin and all or substantially all of the FR residues are those of a bovine immunoglobulin sequence. The bovinized antibody may also comprise at least a portion of an immunoglobulin constant region (Fc), typically that of a bovine immunoglobulin.

As used herein, the term “immunoadhesin” designates antibody-like molecules which combine the binding domain of a heterologous “adhesin” protein (e.g., a receptor, ligand or enzyme) with an immunoglobulin constant domain. Structurally, immunoadhesins comprise a fusion of the adhesin amino acid sequence with the desired binding specificity which is other than the antigen recognition and binding site (antigen combining site) of an antibody (i.e., is “heterologous”) with an immunoglobulin constant domain sequence.

As used herein, the term “ligand binding domain” refers to any native receptor or any region or derivative thereof retaining at least a qualitative ligand binding ability of a corresponding native receptor. In certain embodiments, the receptor is from a cell-surface polypeptide having an extracellular domain that is homologous to a member of the immunoglobulin supergene family. Other receptors, which are not members of the immunoglobulin supergene family but are nonetheless specifically covered by this definition, are receptors for cytokines, and in particular receptors with tyrosine kinase activity (receptor tyrosine kinases), members of the hematopoietin and nerve growth factor receptor superfamilies, and cell adhesion molecules (e.g., E-, L-, and P-selectins).

As used herein, the term “receptor binding domain” refers to any native ligand for a receptor, including, e.g., cell adhesion molecules, or any region or derivative of such native ligand retaining at least a qualitative receptor binding ability of a corresponding native ligand.

As used herein, an “isolated” polypeptide is one that has been identified and separated and/or recovered from a component of its natural environment. Contaminant components of its natural environment are materials that would interfere with diagnostic or therapeutic uses for the polypeptide, and may include enzymes, hormones, and other proteinaceous or non-proteinaceous solutes. In certain embodiments, the isolated polypeptide is purified (1) to greater than 95% by weight of polypeptides as determined by the Lowry method, and preferably, more than 99% by weight, (2) to a degree sufficient to obtain at least 15 residues of N-terminal or internal amino acid sequence by use of a spinning cup sequenator, or (3) to homogeneity by SDS-page under reducing or nonreducing conditions using Coomassie blue or silver stain. Isolated polypeptide includes the polypeptide in situ within recombinant cells since at least one component of the polypeptide's natural environment will not be present. Ordinarily, however, isolated polypeptide will be prepared by a least one purification step.

As used herein, the term “disorder” and “disease” are used interchangeably to refer to any condition that would benefit from treatment with a variant polypeptide (a polypeptide comprising a variant Fc region of the invention), including chronic and acute disorders or diseases (e.g., pathological conditions that predispose a patient to a particular disorder).

As used herein, the term “receptor” refers to a polypeptide capable of binding at least one ligand. The preferred receptor is a cell-surface or soluble receptor having an extracellular ligand-binding domain and, optionally, other domains (e.g., transmembrane domain, intracellular domain and/or membrane anchor). A receptor to be evaluated in an assay described herein may be an intact receptor or a fragment or derivative thereof (e.g. a fusion protein comprising the binding domain of the receptor fused to one or more heterologous polypeptides). Moreover, the receptor to be evaluated for its binding properties may be present in a cell or isolated and optionally coated on an assay plate or some other solid phase or labeled directly and used as a probe.

As used herein a variant polypeptide that knocks out, or knocks down, antibody-dependent cell-mediated cytotoxicity (ADCC), antibody-dependent cellular phagocytosis (ADCP) and complement-dependent cytotoxicity (CDC) in the presence of bovine effector cells compared to parent antibody is one which in vitro or in vivo is substantially less active at mediating ADCC, ADCP and/or CDC, when the amounts of variant polypeptide and parent antibody used in the assay are essentially the same. For example, such a variant causes a lower, preferably negligible, amount of target cell lysis or phagocytosis in a given ADCC, ADCP or CDC assay than the parent polypeptide in an identical ADCC assay. Such variants may be identified, for example, using an ADCC, ADCP or CDC assay, but other assays or methods for determining ADCC, ADCP or CDC activity may also be employed (e.g., animal models). In preferred embodiments, the variant polypeptide is about 100, 75, 50, or 25 percent less active at mediating ADCC, ADCP and CDC than the parent polypeptide.

### Bovine Wildtype IgG

Bovine IgGs are well known in the art and fully described in, for example, Symons et al., 1989, Mol. Immunol., vol. 26(9), pages 841-850; Kacskovics et al., 1996, Mol. Immunol., vol. 33(2), pages 189-195; Saini et al., 2007, Scand. J. Immunol., vol. 65(1), pages 32-38; and Rabbani et al., 1997, Immunogenetics, vol. 46(4), pages 326-331.

In one embodiment, bovine IgG is IgG1. In another embodiment, bovine IgG is IgG2. In yet another embodiment, bovine IgG is IgG3. In one example, IgG1 is IgG1a, IgG1b, IgG1c, or IgG1d. In another example, IgG2 is IgG2a or IgG2b. In yet another example, IgG3 is IgG3a or IgG3b.

The amino acid and nucleic acid sequences of IgG1a, IgG1b, IgG1c, IgG1d, IgG2a, IgG2b, IgG3a, and IgG3b are also well known in the art.

In one example, IgG of the invention comprises a constant domain, for example, CH1, CH2, or CH3 domains, or a combination thereof. In another example, the constant domain of the invention comprises Fc region, including, for example, CH2 or CH3 domains or a combination thereof.

In a particular example, the wild-type constant domain comprises any one of the amino acid sequences set forth in SEQ ID NOs.: 1-8. In a particular embodiment, the wild-type constant domain of IgG1a, IgG1b, IgG1c, IgG1d, IgG2a, IgG2b, IgG3a, and IgG3b comprises the amino acid sequence set forth in SEQ ID NO.: 1, 2, 3, 4, 5, 6, 7, and 8, respectively. In some embodiments, the wild-type IgG constant domain is a homologue, a variant, an isomer, or a functional fragment of any one of SEQ ID NOs.: 1-8, but without any mutation described herein. Each possibility represents a separate embodiment of the present invention. For example, in one embodiment, in a particular embodiment, the wild-type constant domain of IgG2a comprises the amino acid sequence set forth in SEQ ID NO.: 9.

IgGs contant domains also include polypeptides with amino acid sequences substantially similar to the amino acid sequence of the heavy and/or light chain. Substantially the same amino acid sequence is defined herein as a sequence with at least 70%, 75%, 80%, 85%, 90%, 95%, or 99% identity to a compared amino acid sequence, as determined by the FASTA search method in accordance with Pearson and Lipman, Proc. Natl. Acad. Sci. USA 85:2444-2448 (1988).

The present invention also includes nucleic acid molecules that encode IgGs or portion thereof, described herein. In one embodiment, the nucleic acids may encode an antibody heavy chain comprising, for example, CH1, CH2, CH3 regions, or a combination thereof. In another embodiment, the nucleic acids may encode an antibody heavy chain comprising, for example, any one of the VH regions or a portion thereof, or any one of the VH CDRs, including any variants thereof. The invention also includes nucleic acid molecules that encode an antibody light chain comprising, for example, any one of the CL regions or a portion thereof, any one of the VL regions or a portion thereof or any one of the VL CDRs, including any variants thereof. In certain embodiments, the nucleic acid encodes both a heavy and light chain, or portions thereof.

The amino acid sequence of the wild-type constant domain set forth in SEQ ID NO.: 1, 2, 3, 4, 5, 6, 7, 8, or 9 is encoded by its corresponding nucleic acid sequence.

### Modified Bovine IgG

The inventors of the instant application have found that substituting the amino acid residue at position 216, 234, 235, 237, 270, 329, 330, 331, 432, 434, 437, or 433 with another amino acid surprisingly and unexpectedly exhibited a desired effect. The term, position, as used herein, refers to a position numbered according to the Eu index as in Kabat (Kabat et al., Sequences of Proteins of Immunological Interest, 5th Ed. Public Health Service, National Institutes of Health, Bethesda, Md. (1991)). In one embodiment, the desired effect is eliminating or reducing complement-dependent cytotoxicity, relative to an IgG having the wild-type bovine IgG constant domain. In another embodiment, the desired effect is eliminating or reducing antibody-dependent cellular phagocytosis, relative to an IgG having the wild-type bovine IgG constant domain. In yet another embodiment, the desired effect is eliminating or reducing the binding of the IgG to Fc gamma receptor (bFcgR).

In one embodiment, the invention provides a modified IgG comprising: a bovine IgG constant domain comprising at least one amino acid substitution relative to a wild-type bovine IgG constant domain, wherein the substitution is at amino acid residue 216, 234, 235, 237, 270, 329, 330, 331, 432, 434, 437, or 433, numbered according to the Eu index as in Kabat. The amino acid at these positions can be substituted with any other amino acid. Examples of substitution amino acid includes, for example, but not limited to, asparagine, histidine, serine, alanine, phenylalanine, glycine, isoleucine, lysine, leucine, methionine, glutamine, arginine, threonine, valine, tryptophan, tyrosine, cysteine, aspartic acid, glutamic acid, and proline. In some embodiments, the substitution amino acid is a non-natural amino acid.

The modified bovine IgG of the invention can be any suitable bovine IgG, known to one of skilled in the art. Examples of the modified bovine IgG include a modified variant of IgG1 (e.g., IgG1a, IgG1b, IgG1c, or IgG1d), IgG2 (e.g., IgG2a or IgG2b), or IgG3 (IgG3a or IgG3b).

### IgG1

In one exemplary embodiment, the modified bovine IgG is a modified bovine IgG1, including, for example, a modified IgG1a, a modified IgG1b, a modified IgG1c, or a modified IgG1d.

In one embodiment, the invention provides a modified IgG1 comprising: a bovine IgG1 constant domain comprising at least one amino acid substitution relative to a wild-type bovine IgG1 constant domain, wherein the substitution is at amino acid residue 329, 330, 331, or a combination thereof, and wherein the amino acid residue position is numbered according to the Eu index as in Kabat. The amino acid residue at position 329, 330, or 331 can be substituted with any other amino acid. In a particular embodiment, the substitution is a replacement with serine. Specifically, in one example, the substitution is a substitution of proline at position 329 with serine (P329S), alanine at position 330 with serine (A330S), or proline at position 331 with serine (P331S). In some embodiment, the modified bovine IgG1 constant domain comprises one or more of substitutions P329S, A330S, and P331S.

In one aspect, the modified bovine IgG1 constant domain comprises a PAP to SAP mutation, PAP to SAS mutation, SS mutation, Winter site mutation, or a combination thereof. The PAP to SAP mutation includes a substitution of proline at position 329 with serine (P329S). The SS mutation includes a substitution of alanine at position 330 with serine (A330S) and a substitution of proline at position 331 with serine (P331S).

The Winter site may include a substitution at amino acid residue 234, 235, 237, or a combination thereof. Accordingly, in another embodiment, the invention provides a modified IgG1 comprising: a bovine IgG1 constant domain comprising at least one amino acid substitution relative to a wild-type bovine IgG1 constant domain, wherein the substitution is at amino acid residue 234, 235, or 237, or a combination thereof, and wherein the amino acid residue position is numbered according to the Eu index as in Kabat. The amino acid residue at position 234, 235, or 237 can be substituted with any other amino acid. In a particular embodiment, the substitution is a replacement with alanine. Specifically, in one example, the substitution is a substitution of proline at position 234 with alanine (P234A), leucine at position 235 with alanine (L235A), or glycine at position 235 with alanine (G237A). In some embodiment, the modified bovine IgG1 constant domain comprises one or more of substitutions P234A, L235A, and G237A.

In an exemplary embodiment, the bovine IgG1 constant domain comprises one or more of substitutions P329S, A330S, P331S, P234A, L235A, and G237A.

In one aspect, the modified bovine IgG1 constant domain comprises a substitution in an amino acid residue of the DP site. Accordingly, in another embodiment, the invention provides a modified IgG1 comprising: a bovine IgG1 constant domain comprising at least one amino acid substitution relative to a wild-type bovine IgG1 constant domain, wherein the substitution is at amino acid residue 216, 270, or a combination thereof, and wherein the amino acid residue position is numbered according to the Eu index as in Kabat. The amino acid residue at position 216 or 270 can be substituted with any other amino acid. In a particular embodiment, the substitution is a replacement with glutamic acid. Specifically, in one example, the substitution is a substitution of aspartic acid at position 216 with glutamic acid (D216E) or aspartic acid at position 270 with glutamic acid (D270E). In some embodiment, the modified bovine IgG1 constant domain comprises one or more of substitutions D216E and D270E.

In an exemplary embodiment, the bovine IgG1 constant domain comprises one or more of substitutions P329S, A330S, P331S, P234A, L235A, G237A, D216E and D270E.

In another embodiment, the invention provides a modified IgG1 comprising: a bovine IgG1 constant domain comprising at least one amino acid substitution relative to a wild-type bovine IgG1 constant domain, wherein said substitution is at amino acid residue 432, 434, 437 or a combination thereof, and wherein the amino acid residue position is numbered according to the Eu index as in Kabat. The amino acid residue at position 432, 434, or 437 can be substituted with any other amino acid. In a particular embodiment, the substitution is a replacement with alanine. Specifically, in one example, the substitution is a substitution of leucine at position 432 with alanine (L432A), asparagine at position 434 with alanine (N434A), threonine at position 437 with alanine (T437A). In some embodiment, the modified bovine IgG1 constant domain comprises one or more of substitutions L432A, N434A, and T437A.

In another exemplary embodiment, the bovine IgG1 constant domain comprises one or more of substitutions P329S, A330S, P331S, P234A, L235A, G237A, D216E, D270E, L432A, N434A, and T437A.

### IgG2

In another exemplary embodiment, the modified bovine IgG is a modified bovine IgG2, including, for example, a modified IgG2a or a modified IgG2b. The modified bovine IgG2 may comprise SS mutation, which includes a substitution of alanine at position 330 with serine (A330S) and a substitution of proline at position 331 with serine (P331S). In one embodiment, the invention provides a modified IgG2 comprising: a bovine IgG2 constant domain comprising at least one amino acid substitution relative to a wild-type bovine IgG2 constant domain, wherein the substitution is at amino acid residue 330, 331, or a combination thereof, and wherein the amino acid residue position is numbered according to the Eu index as in Kabat. The amino acid residue at position 330, or 331 can be substituted with any other amino acid. In a particular embodiment, the substitution is a replacement with serine. Specifically, in one example, the substitution is a substitution of alanine at position 330 with serine (A330S) or proline at position 331 with serine (P331S). In some embodiment, the modified bovine IgG2 constant domain comprises one or more of substitutions A330S and P331S.

In another embodiment, the invention provides a modified IgG2 comprising: a bovine IgG2 constant domain comprising at least one amino acid substitution relative to a wild-type bovine IgG2 constant domain, wherein the substitution is at amino acid residue 432, 434, 437 or a combination thereof, and wherein the amino acid residue position is numbered according to the Eu index as in Kabat. The amino acid residue at position 432, 434, or 437 can be substituted with any other amino acid. In a particular embodiment, the substitution is a replacement with alanine. Specifically, in one example, the substitution is a substitution of leucine at position 432 with alanine (L432A), asparagine at position 434 with alanine (N434A), or methionine at position 437 with alanine (M437A). In some embodiment, the modified bovine IgG2 constant domain comprises one or more of substitutions L432A, N434A, and M437A.

In another exemplary embodiment, the bovine IgG2 constant domain comprises one or more of substitutions A330S, L432A, N434A, and M437A.

### IgG3

In another exemplary embodiment, the modified bovine IgG is a modified bovine IgG3, including, for example, a modified IgG3a or a modified IgG3b.

In one embodiment, the invention provides a modified IgG3 comprising: a bovine IgG3 constant domain comprising at least one amino acid substitution relative to a wild-type bovine IgG3 constant domain, wherein the substitution is at amino acid residue 329, 330, 331, or a combination thereof, and wherein the amino acid residue position is numbered according to the Eu index as in Kabat. The amino acid residue at position 329, 330, or 331 can be substituted with any other amino acid. In a particular embodiment, the substitution is a replacement with serine. Specifically, in one example, the substitution is a substitution of proline at position 329 with serine (P329S), alanine at position 330 with serine (A330S), or proline at position 331 with serine (P331S). In some embodiment, the modified bovine IgG3 constant domain comprises one or more of substitutions P329S, A330S, and P331S.

In one aspect, the modified bovine IgG3 constant domain comprises a PAP to SAP mutation, PAP to SAS mutation, SS mutation, Winter site mutation, or a combination thereof. As discussed above, the PAP to SAP mutation includes a substitution of proline at position 329 with serine (P329S); the PAP to SAS mutation includes a substitution of proline at position 331 with serine (P331S); and the SS mutation includes a substitution of alanine at position 330 with serine (A330S) in combination with a substitution of proline at position 331 with serine (P331S).

As discussed above, the Winter site may include a substitution at amino acid residue 234, 235, 237, or a combination thereof. Accordingly, in another embodiment, the invention provides a modified IgG3 comprising: a bovine IgG3 constant domain comprising at least one amino acid substitution relative to a wild-type bovine IgG3 constant domain, wherein said substitution is at amino acid residue 234, 235, or 237, or a combination thereof, and wherein the amino acid residue position is numbered according to the Eu index as in Kabat. The amino acid residue at position 234, 235, or 237 can be substituted with any other amino acid. In a particular embodiment, the substitution is a replacement with alanine. Specifically, in one example, the substitution is a substitution of proline at position 234 with alanine (P234A), leucine at position 235 with alanine (L235A), or glycine at position 235 with alanine (G237A). In some embodiment, the modified bovine IgG3 constant domain comprises one or more of substitutions P234A, L235A, and G237A.

In an exemplary embodiment, the bovine IgG3 constant domain comprises one or more of substitutions P329S, A330S, P331S, P234A, L235A, and G237A.

In one aspect, the modified bovine IgG3 constant domain comprises a substitution in an amino acid residue of the DP site. Accordingly, in another embodiment, the invention provides a modified IgG3 comprising: a bovine IgG3 constant domain comprising at least one amino acid substitution relative to a wild-type bovine IgG3 constant domain, wherein the substitution is at amino acid residue 270, and wherein the amino acid residue position is numbered according to the Eu index as in Kabat. The amino acid residue at position 270 can be substituted with any other amino acid. In a particular embodiment, the substitution is a replacement with glutamic acid. Specifically, in one example, the substitution is a substitution of aspartic acid at position aspartic acid at position 270 with glutamic acid (D270E).

In an exemplary embodiment, the bovine IgG3 constant domain comprises one or more of substitutions P329S, A330S, P331S, P234A, L235A, G237A, and D270E.

In another embodiment, the invention provides a modified IgG3 comprising: a bovine IgG3 constant domain comprising at least one amino acid substitution relative to a wild-type bovine IgG3 constant domain, wherein the substitution is at amino acid residue 432, 434, 437 or a combination thereof, and wherein the amino acid residue position is numbered according to the Eu index as in Kabat. The amino acid residue at position 432, 434, or 437 can be substituted with any other amino acid. In a particular embodiment, the substitution is a replacement with alanine. Specifically, in one example, the substitution is a substitution of leucine at position 432 with alanine (L432A), asparagine at position 434 with alanine (N434A), or lysine at position 437 with alanine (K437A). In some embodiment, the modified bovine IgG1 constant domain comprises one or more of substitutions L432A, N434A, and K437A.

In another exemplary embodiment, the bovine IgG3 constant domain comprises one or more of substitutions P329S, A330S, P331S, P234A, L235A, G237A, D270E, L432A, N434A, and K437A.

In yet another embodiment, the invention provides a modified IgG3 comprising: a bovine IgG3 constant domain comprising at least one amino acid substitution relative to a wild-type bovine IgG3 constant domain, wherein said substitution is at amino acid residue 433, and wherein the amino acid residue position is numbered according to the Eu index as in Kabat. The amino acid residue at position 433 can be substituted with any other amino acid. In a particular embodiment, the substitution is a replacement with histidine. Specifically, in one example, the substitution is a substitution of arginine at position 433 with histidine (R433H).

In yet another exemplary embodiment, the bovine IgG3 constant domain comprises one or more of substitutions P329S, A330S, P331S, P234A, L235A, G237A, D270E, L432A, N434A, K437A, and R433H.

In a particular example, the mutant IgG1 constant domain of the invention comprises any one of the amino acid sequences set forth in SEQ ID NOs.: 10-12 and 14-18. In some embodiments, the mutant IgG1 constant domain is a homologue, a variant, an isomer, or a functional fragment of any one of SEQ ID NOs.: 10-12 and 14-18, but with mutation of the invention described herein. Each possibility represents a separate embodiment of the present invention.

In another example, the mutant IgG2 constant domain of the invention comprises any one of the amino acid sequences set forth in SEQ ID NOs.: 19-25. In some embodiments, the mutant IgG2 constant domain is a homologue, a variant, an isomer, or a functional fragment of any one of SEQ ID NOs.: 19-25, but with mutation of the invention described herein. Each possibility represents a separate embodiment of the present invention.

In a particular example, the mutant IgG3 constant domain of the invention comprises any one of the amino acid sequences set forth in SEQ ID NOs.: 27-31. In some embodiments, the mutant IgG3 constant domain is a homologue, a variant, an isomer, or a functional fragment of any one of SEQ ID NOs.: 27-31, but with mutation of the invention described herein. Each possibility represents a separate embodiment of the present invention.

The amino acid sequence of the mutant constant domain set forth in SEQ ID NO.: 10-12, 14-25, and 27-31 is encoded by its corresponding mutant nucleic acid sequence.

### Methods for Making Antibody Molecules of the Invention

Methods for making antibody molecules are well known in the art and fully described in U.S. Pat. Nos. 8,394,925; 8,088,376; 8,546,543; 10,336,818; and 9,803,023 and U.S. Patent Application Publication 20060067930, which are incorporated by reference herein in their entirety. Any suitable method, process, or technique, known to one of skilled in the art, can be used. An antibody molecule having a variant Fc region of the invention may be generated according to the methods well known in the art. In some embodiments, the variant Fc region can be fused to a heterologous polypeptide of choice, such as an antibody variable domain or binding domain of a receptor or ligand.

With the advent of methods of molecular biology and recombinant technology, a person of skilled in the art can produce antibody and antibody-like molecules by recombinant means and thereby generate gene sequences that code for specific amino acid sequences found in the polypeptide structure of the antibodies. Such antibodies can be produced by either cloning the gene sequences encoding the polypeptide chains of said antibodies or by direct synthesis of said polypeptide chains, with assembly of the synthesized chains to form active tetrameric (H2L2) structures with affinity for specific epitopes and antigenic determinants. This has permitted the ready production of antibodies having sequences characteristic of neutralizing antibodies from different species and sources.

Regardless of the source of the antibodies, or how they are recombinantly constructed, or how they are synthesized, in vitro or in vivo, using transgenic animals, large cell cultures of laboratory or commercial size, using transgenic plants, or by direct chemical synthesis employing no living organisms at any stage of the process, all antibodies have a similar overall 3 dimensional structure. This structure is often given as H2L2 and refers to the fact that antibodies commonly comprise two light (L) amino acid chains and 2 heavy (H) amino acid chains. Both chains have regions capable of interacting with a structurally complementary antigenic target. The regions interacting with the target are referred to as “variable” or ‘V” regions and are characterized by differences in amino acid sequence from antibodies of different antigenic specificity. The variable regions of either H or L chains contain the amino acid sequences capable of specifically binding to antigenic targets.

As used herein, the term “antigen binding region” refers to that portion of an antibody molecule which contains the amino acid residues that interact with an antigen and confer on the antibody its specificity and affinity for the antigen. The antibody binding region includes the “framework” amino acid residues necessary to maintain the proper conformation of the antigen-binding residues. Within the variable regions of the H or L chains that provide for the antigen binding regions are smaller sequences dubbed “hypervariable” because of their extreme variability between antibodies of differing specificity. Such hypervariable regions are also referred to as “complementarity determining regions” or “CDR” regions. These CDR regions account for the basic specificity of the antibody for a particular antigenic determinant structure.

The CDRs represent non-contiguous stretches of amino acids within the variable regions but, regardless of species, the positional locations of these critical amino acid sequences within the variable heavy and light chain regions have been found to have similar locations within the amino acid sequences of the variable chains. The variable heavy and light chains of all antibodies each have three CDR regions, each non-contiguous with the others. In all mammalian species, antibody peptides contain constant (i.e., highly conserved) and variable regions, and, within the latter, there are the CDRs and the so-called “framework regions” made up of amino acid sequences within the variable region of the heavy or light chain but outside the CDRs.

The present invention further provides a vector including at least one of the nucleic acids described above. Because the genetic code is degenerate, more than one codon can be used to encode a particular amino acid. Using the genetic code, one or more different nucleotide sequences can be identified, each of which would be capable of encoding the amino acid. The probability that a particular oligonucleotide will, in fact, constitute the actual encoding sequence can be estimated by considering abnormal base pairing relationships and the frequency with which a particular codon is actually used (to encode a particular amino acid) in eukaryotic or prokaryotic cells expressing an antibody or portion. Such “codon usage rules” are disclosed by Lathe, et al., 183 J. Molec. Biol. 1-12 (1985). Using the “codon usage rules” of Lathe, a single nucleotide sequence, or a set of nucleotide sequences that contains a theoretical “most probable” nucleotide sequence capable of encoding bovine IgG sequences can be identified. It is also intended that the antibody coding regions for use in the present invention could also be provided by altering existing antibody genes using standard molecular biological techniques that result in variants of the antibodies and peptides described herein. Such variants include, but are not limited to deletions, additions and substitutions in the amino acid sequence of the antibodies or peptides.

For example, one class of substitutions is conservative amino acid substitutions. Such substitutions are those that substitute a given amino acid in a bovine antibody peptide by another amino acid of like characteristics. Typically seen as conservative substitutions are the replacements, one for another, among the aliphatic amino acids Ala, Val, Leu, and Ile; interchange of the hydroxyl residues Ser and Thr, exchange of the acidic residues Asp and Glu, substitution between the amide residues Asn and Gin, exchange of the basic residues Lys and Arg, replacements among the aromatic residues Phe, Tyr, and the like. Guidance concerning which amino acid changes are likely to be phenotypically silent is found in Bowie et al., 247 Science 1306-10 (1990).

Variant bovine antibodies or peptides may be fully functional or may lack function in one or more activities. Fully functional variants typically contain only conservative variations or variations in non-critical residues or in non-critical regions. Functional variants can also contain substitution of similar amino acids that result in no change or an insignificant change in function. Alternatively, such substitutions may positively or negatively affect function to some degree. Non-functional variants typically contain one or more non-conservative amino acid substitutions, deletions, insertions, inversions, or truncation or a substitution, insertion, inversion, or deletion in a critical residue or critical region.

Amino acids that are essential for function can be identified by methods known in the art, such as site-directed mutagenesis or alanine-scanning mutagenesis. Cunningham et al., 244 Science 1081-85 (1989). The latter procedure introduces single alanine mutations at every residue in the molecule. The resulting mutant molecules are then tested for biological activity such as epitope binding or in vitro ADCC activity. Sites that are critical for ligand-receptor binding can also be determined by structural analysis such as epitope mapping (e.g., HDX), crystallography, nuclear magnetic resonance, or photoaffinity labeling. Smith et al., 224 J. Mol. Biol. 899-904 (1992); de Vos et al., 255 Science 306-12 (1992).

Moreover, polypeptides often contain amino acids other than the twenty “naturally occurring” amino acids. Further, many amino acids, including the terminal amino acids, may be modified by natural processes, such as processing and other post-translational modifications, or by chemical modification techniques well known in the art. Known modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Such modifications are well known to those of skill in the art and have been described in great detail in the scientific literature. Several particularly common modifications, glycosylation, lipid attachment, sulfation, gamma-carboxylation of glutamic acid residues, hydroxylation and ADP ribosylation, for instance, are described in most basic texts, such as Proteins-Structure and Molecular Properties (2nd ed., T. E. Creighton, W. H. Freeman & Co., N.Y., 1993). Many detailed reviews are available on this subject, such as by Wold, Posttranslational Covalent Modification of proteins, 1-12 (Johnson, ed., Academic Press, N.Y., 1983); Seifter et al. 182 Meth. Enzymol. 626-46 (1990); and Rattan et al. 663 Ann. NY Acad. Sci. 48-62 (1992).

In another aspect, the invention provides antibody derivatives. A “derivative” of an antibody contains additional chemical moieties not normally a part of the protein. Covalent modifications of the protein are included within the scope of this invention. Such modifications may be introduced into the molecule by reacting targeted amino acid residues of the antibody with an organic derivatizing agent that is capable of reacting with selected side chains or terminal residues. For example, derivatization with bifunctional agents, well-known in the art, is useful for cross-linking the antibody or fragment to a water-insoluble support matrix or to other macromolecular carriers.

Derivatives also include radioactively labeled monoclonal antibodies that are labeled. For example, with radioactive iodine (251,1311), carbon (4C), sulfur (35S), indium, tritium (H³) or the like; conjugates of monoclonal antibodies with biotin or avidin, with enzymes, such as horseradish peroxidase, alkaline phosphatase, beta-D-galactosidase, glucose oxidase, glucoamylase, carboxylic acid anhydrase, acetylcholine esterase, lysozyme, malate dehydrogenase or glucose 6-phosphate dehydrogenase; and also conjugates of monoclonal antibodies with bioluminescent agents (such as luciferase), chemoluminescent agents (such as acridine esters) or fluorescent agents (such as phycobiliproteins).

Another derivative bifunctional antibody of the invention is a bispecific antibody, generated by combining parts of two separate antibodies that recognize two different antigenic groups. This may be achieved by crosslinking or recombinant techniques. Additionally, moieties may be added to the antibody or a portion thereof to increase half-life in vivo (e.g., by lengthening the time to clearance from the blood stream. Such techniques include, for example, adding PEG moieties (also termed pegylation), and are well-known in the art. See U.S. Patent. Appl. Pub. No. 20030031671.

In some embodiments, the nucleic acids encoding a subject antibody are introduced directly into a host cell, and the cell is incubated under conditions sufficient to induce expression of the encoded antibody. After the subject nucleic acids have been introduced into a cell, the cell is typically incubated, normally at 37° C., sometimes under selection, for a period of about 1-24 hours in order to allow for the expression of the antibody. In one embodiment, the antibody is secreted into the supernatant of the media in which the cell is growing. Traditionally, monoclonal antibodies have been produced as native molecules in murine hybridoma lines. In addition to that technology, the present invention provides for recombinant DNA expression of the antibodies. This allows the production of antibodies, as well as a spectrum of antibody derivatives and fusion proteins in a host species of choice.

A nucleic acid sequence encoding at least one antibody, portion or polypeptide of the invention may be recombined with vector DNA in accordance with conventional techniques, including blunt-ended or staggered-ended termini for ligation, restriction enzyme digestion to provide appropriate termini, filling in of cohesive ends as appropriate, alkaline phosphatase treatment to avoid undesirable joining, and ligation with appropriate ligases. Techniques for such manipulations are disclosed, e.g., by Maniatis et al., MOLECULAR CLONING, LAB. MANUAL, (Cold Spring Harbor Lab. Press, N Y, 1982 and 1989), and Ausubel et al. 1993 supra, may be used to construct nucleic acid sequences which encode an antibody molecule or antigen binding region thereof.

A nucleic acid molecule, such as DNA, is said to be “capable of expressing” a polypeptide if it contains nucleotide sequences which contain transcriptional and translational regulatory information and such sequences are “operably linked” to nucleotide sequences which encode the polypeptide. An operable linkage is a linkage in which the regulatory DNA sequences and the DNA sequence sought to be expressed are connected in such a way as to permit gene expression as peptides or antibody portions in recoverable amounts. The precise nature of the regulatory regions needed for gene expression may vary from organism to organism, as is well known in the analogous art. See, e.g., Sambrook et al., 2001 supra; Ausubel et al., 1993 supra.

The present invention accordingly encompasses the expression of an antibody or peptide, in either prokaryotic or eukaryotic cells. Suitable hosts include bacterial or eukaryotic hosts including bacteria, yeast, insects, fungi, bird and mammalian cells either in vivo, or in situ, or host cells of mammalian, insect, bird or yeast origin. The mammalian cell or tissue may be of human, primate, hamster, rabbit, rodent, cow, pig, sheep, horse, goat, dog or cat origin. Any other suitable mammalian cell, known in the art, may also be used.

In one embodiment, the nucleotide sequence of the invention will be incorporated into a plasmid or viral vector capable of autonomous replication in the recipient host. Any of a wide variety of vectors may be employed for this purpose. See, e.g., Ausubel et al., 1993 supra. Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

Example prokaryotic vectors known in the art include plasmids such as those capable of replication in E. coli (such as, for example, pBR322, CoIE1, pSC101, pACYC 184, .pi.vX). Such plasmids are, for example, disclosed by Maniatis et al., 1989 supra; Ausubel et al, 1993 supra. Bacillus plasmids include pC194, pC221, pT127, etc. Such plasmids are disclosed by Gryczan, in THE MOLEC. BIO. OF THE BACILLI 307-329 (Academic Press, N Y, 1982). Suitable Streptomyces plasmids include p1J101 (Kendall et al., 169 J. Bacteriol. 4177-83 (1987), and Streptomyces bacteriophages such as phLC31 (Chater et al., in SIXTH INT'L SYMPOSIUM ON ACTINOMYCETALES BIO. 45-54 (Akademiai Kaido, Budapest, Hungary 1986). Pseudomonas plasmids are reviewed in John et al., 8 Rev. Infect. Dis. 693-704 (1986); lzaki, 33 Jpn. J. Bacteriol. 729-42 (1978); and Ausubel et al., 1993 supra.

Alternatively, gene expression elements useful for the expression of cDNA encoding antibodies or peptides include, but are not limited to, (a) viral transcription promoters and their enhancer elements, such as the SV40 early promoter (Okayama et al., 3 Mol. Cell. Biol. 280 (1983), Rous sarcoma virus LTR (Gorman et al., 79 Proc. Natl. Acad. Sci., USA 6777 (1982), and Moloney murine leukemia virus LTR (Grosschedl et al., 41 Cell 885 (1985); (b) splice regions and polyadenylation sites such as those derived from the SV40 late region (Okayarea et al., 1983), and (c) polyadenylation sites such as in SV40 (Okayama et al., 1983).

Immunoglobulin cDNA genes can be expressed as described by Weidle et al., 51 Gene 21 (1987), using as expression elements the SV40 early promoter and its enhancer, the mouse immunoglobulin H chain promoter enhancers, SV40 late region mRNA splicing, rabbit S-globin intervening sequence, immunoglobulin and rabbit S-globin polyadenylation sites, and SV40 polyadenylation elements. For immunoglobulin genes comprised of part cDNA, part genomic DNA (Whittle et al., 1 Protein Engin. 499 (1987)), the transcriptional promoter can be human cytomegalovirus, the promoter enhancers can be cytomegalovirus and mouse/human immunoglobulin, and mRNA splicing and polyadenylation regions can be the native chromosomal immunoglobulin sequences.

In one embodiment, for expression of cDNA genes in rodent cells, the transcriptional promoter is a viral LTR sequence, the transcriptional promoter enhancers are either or both the mouse immunoglobulin heavy chain enhancer and the viral LTR enhancer, the splice region contains an intron of greater than 31 bp, and the polyadenylation and transcription termination regions are derived from the native chromosomal sequence corresponding to the immunoglobulin chain being synthesized. In other embodiments, cDNA sequences encoding other proteins are combined with the above-recited expression elements to achieve expression of the proteins in mammalian cells.

Each fused gene can be assembled in, or inserted into, an expression vector. Recipient cells capable of expressing the immunoglobulin chain gene product are then transfected singly with a peptide or H or L chain-encoding gene, or are co-transfected with H and L chain gene. The transfected recipient cells are cultured under conditions that permit expression of the incorporated genes and the expressed immunoglobulin chains or intact antibodies or fragments are recovered from the culture.

In one embodiment, the fused genes encoding the peptide or H and L chains, or portions thereof are assembled in separate expression vectors that are then used to cotransfect a recipient cell. Alternatively the fused genes encoding the H and L chains can be assembled on the same expression vector. For transfection of the expression vectors and production of the antibody, the recipient cell line may be a myeloma cell. Myeloma cells can synthesize, assemble and secrete immunoglobulins encoded by transfected immunoglobulin genes and possess the mechanism for glycosylation of the immunoglobulin. Myeloma cells can be grown in culture or in the peritoneal cavity of a mouse, where secreted immunoglobulin can be obtained from ascites fluid. Other suitable recipient cells include lymphoid cells such as B lymphocytes of bovine or non-bovine origin, hybridoma cells of bovine or non-bovine origin, or interspecies heterohybridoma cells.

The expression vector carrying an antibody construct or polypeptide of the invention can be introduced into an appropriate host cell by any of a variety of suitable means, including such biochemical means as transformation, transfection, conjugation, protoplast fusion, calcium phosphate-precipitation, and application with polycations such as diethylaminoethyl (DEAE) dextran, and such mechanical means as electroporation, direct microinjection, and microprojectile bombardment. Johnston et al., 240 Science 1538 (1988).

Yeast may provide substantial advantages over bacteria for the production of immunoglobulin H and L chains. Yeasts carry out post-translational peptide modifications including glycosylation. A number of recombinant DNA strategies now exist which utilize strong promoter sequences and high copy number plasmids which can be used for production of the desired proteins in yeast. Yeast recognizes leader sequences of cloned mammalian gene products and secretes peptides bearing leader sequences (i.e., pre-peptides). Hitzman et al., 11th Int'l Conference on Yeast, Genetics & Molec. Biol. (Montpelier, France, 1982).

Yeast gene expression systems can be routinely evaluated for the levels of production, secretion and the stability of peptides, antibodies, fragments and regions thereof. Any of a series of yeast gene expression systems incorporating promoter and termination elements from the actively expressed genes coding for glycolytic enzymes produced in large quantities when yeasts are grown in media rich in glucose can be utilized. Known glycolytic genes can also provide very efficient transcription control signals. For example, the promoter and terminator signals of the phosphoglycerate kinase (PGK) gene can be utilized. A number of approaches can be taken for evaluating optimal expression plasmids for the expression of cloned immunoglobulin cDNAs in yeast. See Vol. II DNA Cloning, 45-66, (Glover, ed.,) IRL Press, Oxford, UK 1985).

Bacterial strains can also be utilized as hosts for the production of antibody molecules or peptides described by this invention. Plasmid vectors containing replicon and control sequences which are derived from species compatible with a host cell are used in connection with these bacterial hosts. The vector carries a replication site, as well as specific genes which are capable of providing phenotypic selection in transformed cells. A number of approaches can be taken for evaluating the expression plasmids for the production of antibodies, fragments and regions or antibody chains encoded by the cloned immunoglobulin cDNAs in bacteria (see Glover, 1985 supra; Ausubel, 1993 supra; Sambrook, 2001 supra; Colligan et al., eds. Current Protocols in Immunology, John Wiley & Sons, NY, N.Y. (1994-2001); Colligan et al., eds. Current Protocols in Protein Science, John Wiley & Sons, NY, N.Y. (1997-2001).

Host mammalian cells may be grown in vitro or in vivo. Mammalian cells provide posttranslational modifications to immunoglobulin protein molecules including leader peptide removal, folding and assembly of Hand L chains, glycosylation of the antibody molecules, and secretion of functional antibody protein. Mammalian cells which can be useful as hosts for the production of antibody proteins, in addition to the cells of lymphoid origin described above, include cells of fibroblast origin, such as Vero (ATCC CRL 81) or CHO-K1 (ATCC CRL 61) cells. Many vector systems are available for the expression of cloned peptides Hand L chain genes in mammalian cells (see Glover, 1985 supra). Different approaches can be followed to obtain complete H2L2 antibodies. It is possible to co-express Hand L chains in the same cells to achieve intracellular association and linkage of Hand L chains into complete tetrameric H2L2 antibodies and/or peptides. The co-expression can occur by using either the same or different plasmids in the same host. Genes for both Hand L chains and/or peptides can be placed into the same plasmid, which is then transfected into cells, thereby selecting directly for cells that express both chains. Alternatively, cells can be transfected first with a plasmid encoding one chain, for example the L chain, followed by transfection of the resulting cell line with an H chain plasmid containing a second selectable marker. cell lines producing peptides and/or H2L2 molecules via either route could be transfected with plasmids encoding additional copies of peptides, H, L, or H plus L chains in conjunction with additional selectable markers to generate cell lines with enhanced properties, such as higher production of assembled H2L2 antibody molecules or enhanced stability of the transfected cell lines.

For long-term, high-yield production of recombinant antibodies, stable expression may be used. For example, cell lines, which stably express the antibody molecule may be engineered. Rather than using expression vectors which contain viral origins of replication, host cells can be transformed with immunoglobulin expression cassettes and a selectable marker. Following the introduction of the foreign DNA, engineered cells may be allowed to grow for 1-2 days in enriched media, and then are switched to a selective media. The selectable marker in the recombinant plasmid confers resistance to the selection and allows cells to stably integrate the plasmid into a chromosome and grow to form foci which in turn can be cloned and expanded into cell lines. Such engineered cell lines may be particularly useful in screening and evaluation of compounds/components that interact directly or indirectly with the antibody molecule.

Once an antibody of the invention has been produced, it may be purified by any method known in the art for purification of an immunoglobulin molecule, for example, by chromatography (e.g., ion exchange, affinity, particularly affinity for the specific antigen after Protein A, and sizing column chromatography), centrifugation, differential solubility, or by any other standard technique for the purification of proteins. In many embodiments, antibodies are secreted from the cell into culture medium and harvested from the culture medium.

### Pharmaceutical and Veterinary Applications

The invention also provides a pharmaceutical composition comprising molecules of the invention and one or more pharmaceutically acceptable carriers. More specifically, the invention provides for a pharmaceutical composition comprising a pharmaceutically acceptable carrier or diluent and, as active ingredient, an antibody or peptide according to the invention.

“Pharmaceutically acceptable carriers” include any excipient which is nontoxic to the cell or animal being exposed thereto at the dosages and concentrations employed. The pharmaceutical composition may include one or additional therapeutic agents.

“Pharmaceutically acceptable” refers to those compounds, materials, compositions, and/or dosage forms which are, within the scope of sound medical judgment, suitable for contact with the tissues of animals without excessive toxicity, irritation, allergic response, or other problem complications commensurate with a reasonable benefit/risk ratio.

Pharmaceutically acceptable carriers include solvents, dispersion media, buffers, coatings, antibacterial and antifungal agents, wetting agents, preservatives, buggers, chelating agents, antioxidants, isotonic agents and absorption delaying agents.

Pharmaceutically acceptable carriers include water; saline; phosphate buffered saline; dextrose; glycerol; alcohols such as ethanol and isopropanol; phosphate, citrate and other organic acids; ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; EDTA; salt forming counterions such as sodium; and/or nonionic surfactants such as TWEEN, polyethylene glycol (PEG), and PLURONICS; isotonic agents such as sugars, polyalcohols such as mannitol and sorbitol, and sodium chloride; as well as combinations thereof.

The pharmaceutical compositions of the invention may be formulated in a variety of ways, including for example, liquid, semi-solid, or solid dosage forms, such as liquid solutions (e.g., injectable and infusible solutions), dispersions or suspensions, liposomes, suppositories, tablets, pills, or powders. In some embodiments, the compositions are in the form of injectable or infusible solutions. The composition can be in a form suitable for intravenous, intraarterial, intramuscular, subcutaneous, parenteral, transmucosal, oral, topical, or transdermal administration. The composition may be formulated as an immediate, controlled, extended or delayed release composition.

The compositions of the invention can be administered either as individual therapeutic agents or in combination with other therapeutic agents. They can be administered alone, but are generally administered with a pharmaceutical carrier selected on the basis of the chosen route of administration and standard pharmaceutical practice. Administration of the antibodies disclosed herein may be carried out by any suitable means, including parenteral injection (such as intraperitoneal, subcutaneous, or intramuscular injection), orally, or by topical administration of the antibodies (typically carried in a pharmaceutical formulation) to an airway surface. Topical administration to an airway surface can be carried out by intranasal administration (e.g., by use of dropper, swab, or inhaler). Topical administration of the antibodies to an airway surface can also be carried out by inhalation administration, such as by creating respirable particles of a pharmaceutical formulation (including both solid and liquid particles) containing the antibodies as an aerosol suspension, and then causing the subject to inhale the respirable particles. Methods and apparatus for administering respirable particles of pharmaceutical formulations are well known, and any conventional technique can be employed.

In some desired embodiments, the antibodies are administered by parenteral injection. For parenteral administration, antibodies or molecules can be formulated as a solution, suspension, emulsion or lyophilized powder in association with a pharmaceutically acceptable parenteral vehicle. For example, the vehicle may be a solution of the antibody or a cocktail thereof dissolved in an acceptable carrier, such as an aqueous carrier such vehicles are water, saline, Ringer's solution, dextrose solution, trehalose or sucrose solution, or 5% serum albumin, 0.4% saline, 0.3% glycine and the like. Liposomes and nonaqueous vehicles such as fixed oils can also be used. These solutions are sterile and generally free of particulate matter. These compositions may be sterilized by conventional, well known sterilization techniques. The compositions may contain pharmaceutically acceptable auxiliary substances as required to approximate physiological conditions such as pH adjusting and buffering agents, toxicity adjustment agents and the like, for example sodium acetate, sodium chloride, potassium chloride, calcium chloride, sodium lactate, etc. The concentration of antibody in these formulations can vary widely, for example from less than about 0.5%, usually at or at least about 1% to as much as 15% or 20% by weight and will be selected primarily based on fluid volumes, viscosities, etc., in accordance with the particular mode of administration selected. The vehicle or lyophilized powder can contain additives that maintain isotonicity (e.g., sodium chloride, mannitol) and chemical stability (e.g., buffers and preservatives). The formulation is sterilized by commonly used techniques. Actual methods for preparing parenterally administrable compositions will be known or apparent to those skilled in the art and are described in more detail in, for example, REMINGTON'S PHARMA. SCI. (15th ed., Mack Pub. Co., Easton, Pa., 1980).

The antibodies or molecules of the invention can be lyophilized for storage and reconstituted in a suitable carrier prior to use. This technique has been shown to be effective with conventional immune globulins. Any suitable lyophilization and reconstitution techniques can be employed. It will be appreciated by those skilled in the art that lyophilization and reconstitution can lead to varying degrees of antibody activity loss and that use levels may have to be adjusted to compensate. The compositions containing the present antibodies or a cocktail thereof can be administered for prevention of recurrence and/or therapeutic treatments for existing disease. Suitable pharmaceutical carriers are described in the most recent edition of REMINGTON'S PHARMACEUTICAL SCIENCES, a standard reference text in this field of art. In therapeutic application, compositions are administered to a subject already suffering from a disease, in an amount sufficient to cure or at least partially arrest or alleviate the disease and its complications.

Effective doses of the compositions of the present invention, for treatment of conditions or diseases as described herein vary depending upon many different factors, including, for example, but not limited to, the pharmacodynamic characteristics of the particular agent, and its mode and route of administration; target site; physiological state of the animal; other medications administered; whether treatment is prophylactic or therapeutic; age, health, and weight of the recipient; nature and extent of symptoms kind of concurrent treatment, frequency of treatment, and the effect desired.

Single or multiple administrations of the compositions can be carried out with dose levels and pattern being selected by the treating veterinarian. In any event, the pharmaceutical formulations should provide a quantity of the antibody(ies) of this invention sufficient to effectively treat the subject.

Treatment dosages may be titrated using routine methods known to those of skill in the art to optimize safety and efficacy.

The pharmaceutical compositions of the invention may include a “therapeutically effective amount.” A “therapeutically effective amount” refers to an amount effective, at dosages and for periods of time necessary, to achieve the desired therapeutic result. A therapeutically effective amount of a molecule may vary according to factors such as the disease state, age, sex, and weight of the individual, and the ability of the molecule to elicit a desired response in the individual. A therapeutically effective amount is also one in which any toxic or detrimental effects of the molecule are outweighed by the therapeutically beneficial effects.

In another aspect, the compositions of the invention can be used, for example, in the treatment of various diseases and disorders in bovine. As used herein, the terms “treat” and “treatment” refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition or those in which the disease or condition is to be prevented.

All patents and literature references cited in the present specification are hereby incorporated by reference in their entirety.

The following examples are provided to supplement the prior disclosure and to provide a better understanding of the subject matter described herein. These examples should not be considered to limit the described subject matter. It is understood that the examples and embodiments described herein are for illustrative purposes only and that various modifications or changes in light thereof will be apparent to persons skilled in the art and are to be included within, and can be made without departing from, the true scope of the invention.

### EXAMPLES

### Example 1

### Bovine IgG Mutants

### Methods

### In Vitro Fc Receptor Binding Assays

Recombinant bovine FcRn (bFcRn), FcgR1, FcgR2, FcgR3, and Fcg2R DNA were codon-optimized for mammalian expression and synthesized based on sequences from NCBI database as in Table 1. DNA was cloned into pcDNA3.1(+) vectors, engineered with a c-terminal 6×His+BAP tag (AGLNDIFEAQKIEWHE; SEQ ID NO.: 113). All FcgRs were transfected (FcRn-α subunit and β-microglobulin were co-transfected) into HEK 293 or Expi-CHO cells and the FcgRs or FcRn complex were purified by IMAC affinity purification via the c-terminal His tag.

The purified FcRs were biotinylated as follows. The purified Fc receptor proteins were dialyzed into 10 mM Tris-HCl, pH 8.0 and concentrated using Amicon Ultra, 10KMWCO (EMD Millipore, Billerica, MA). The Biotin Acceptor Peptide (BAP) AGLNDIFEAQKIEWHE (SEQ ID NO.: 113) which was expressed at the c-terminus of the receptors allowed for transfer of biotin to this stretch of amino acids using the biotin ligase BirA. Biotinylation reactions were carried out as described in the manufacturer protocol (Avidity, LLC, Aurora, CO). The receptors were then dialyzed into PBS to remove residual biotin.

A Biacore SPR binding assay was designed to test the affinity of bovine IgG subclasses and mutants to bFcRn, bFcgR1, bFcgR2, bFcgR3, bFcg2R.

TABLE 1
Sequence references for recombinant bovine Fc receptors

| Bovine FcR | Subunits, subtype, alias | NCBI accession number |
|---|---|---|
| bFcRn | bFcRn alpha subunit | BC102159.1 |
| | β-microglobulin, beta subunit | NM_173893.3 |
| bFcgR1 | bFcgRI | AF162866 |
| bFcgR2 | bFcgRII | X75671.1 |
| bFcgR3 | bFcgRIII | BC112756.1 |
| bFcg2R | | Z37506.1 |

Biacore Method for bFcRn:

Bovine Fc-based antibodies or fusion protein binding affinities to bovine FcRn were determined by surface plasmon resonance (SPR). All reported KD's were measured in Biacore T200 (Cytiva, Marlborough, MA, USA) using SA sensor. Bovine FcRn was captured on the surface of the sensor for a desired surface density. Running buffer of 20 mM MES, 150 mM NaCl, 0.005% Tween 20, 0.5 mg/mL BSA, pH 6 and/or PBS, 0.0005% Tween 20, pH7.4 were used. Various concentrations of bovine CTLA4-Fc fusions or mAbs were titrated in proper running buffer and flowed over the receptor surface. Regeneration was performed with 50 mM Tris-HCl, pH8, 0.005% p20 and 0.5% BSA. Kinetic binding affinity was analyzed using Biacore T200 Evaluation software (Cytiva, Marlborough, MA, USA) with method of double referencing: the reference flow cell was subtracted from the flow cell containing immobilized bovine FcRn and blank runs containing buffer only were subtracted out from all runs. The resulting curve was fitted with the 1:1 binding model. Runs were performed at 25° C.

Method for Biotinylated bFcgR1, bFcgR2 and bFcgR3:

A Biacore SPR binding assay was designed to test the affinity of bovine IgG subclasses to bFcgR1, bFcgR2 and bFcgR3. All reported KD's were measured by Biacore (Cytiva, Marlborough, MA, USA) using series S SA sensor. Biotinylated-bovine FcgR1, R2 and R3 were captured on the sensor surface using a modified SA capture method to reach the desired surface density. 10 mM HEPES, 150 mM NaCl, 3 mM EDTA, 0.05% v/v surfactant P20, pH7.4 buffer was used as the running and titration buffer. Various concentrations of bovine CTLA4-Fc fusions or mAbs were titrated and flowed over the receptor surface and affinities were determined using Biacore T200 Evaluation software (Cytiva, Marlborough, MA, USA) with 1:1 binding model. The method of double referencing has been applied where the reference flow cell was subtracted from the flow cell containing immobilized receptors and blank runs containing buffer only were subtracted out from all runs. Flow cells were regenerated using 10 mM Glycine pH1.5. Runs were performed at 15° C.

Method for bFcg2R:

All reported KD's were measured in Biacore T200 (Cytiva, Marlborough, MA, USA) using Series-S CM5 sensor. Bovine Fcg2R was immobilized on the sensor surface using immobilization buffer by amine coupling (carboxyl group activation by EDC-NGF mixture and deactivate excess reactive groups by Ethanolamine) for a desired surface density. Running buffer of 10 mM HEPES, 150 mM NaCl, 3 mM EDTA, 0.05% v/v surfactant P20, pH7.4 buffer was used. Various concentrations of bovine CTLA4-Fc fusions or mAbs were titrated and flowed over the receptor surface and affinities were determined using Biacore T200 Evaluation software (Cytiva, Marlborough, MA, USA) with 1:1 binding model. The method of double referencing has been applied where the reference flow cell was subtracted from the flow cell containing immobilized receptors and blank runs containing buffer only were subtracted out from all runs. Flow cells were regenerated using 10 mM Glycine pH1.5. Runs were performed at 15° C.

Generation of Fc Fusion Proteins and mAbs

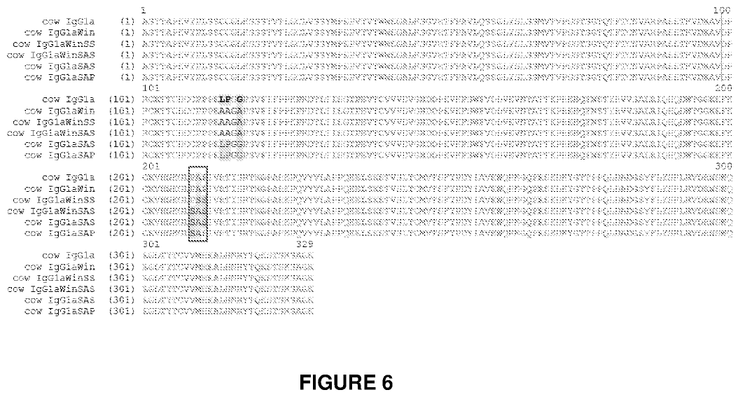

Recombinant CTLA4-Fc fusions were constructed via insertion of the canine CTLA4 gene (NCBI NM_001003106.1) into pcDNA3.1(+) mammalian expression vector containing the bIgG1a (NCBI 1S82409), bIgG2a (sequenced at Zoetis), bIgG3a (NCBI BTU63638) Fc starting just upstream of the heavy chain hinge region as shown in FIGS. 6, 14 and 18, respectively. Recombinant CTLA4-Fc fusions were also constructed for bIgG1b (X16701), bIgG2b (S82407) and or bIgG3b (BTU63639) at identical Fc locations as shown in FIGS. 6, 14, and 18. No additional linkers were required.

Recombinant mAbs with bIgG1a, bIgG1b, bIgG2a, bIgG2b, bIgG3a, and bIgG3b Fc regions were constructed via insertion of VH sequences upstream and in frame with the nucleotides encoding for the constant domains in pcDNA3.1(+) mammalian expression vectors. The constant regions were either bIgG1a (NCBI 1S82409), bIgG1b (X16701), bIgG2a (sequenced at Zoetis), bIgG2b (S82407), bIgG3a (NCBI BTU63638) or bIgG3b (BTU63639). Similarly, light chains were constructed via insertion of VL sequences upstream and in frame with the bovine kappa allele 1 constant region (NCBI HQ213994.1).

Mutations were introduced into the three wildtype subclasses in both the CTLA4 Fc fusion and full mAb formats to knock out binding to FcgRs, knock out CDC and/or ADCP, improve stability, or increase affinity to bFcRn. Mutations were incorporated into various positions on each wildtype plasmid using Agilent's QuikChange II Mutagenesis and associated Agilent primer design tools for single site directed mutagenesis (https://www.agilent.com/store/primerDesignProgram.jsp).

DNA for all CTLA4 fusion and mAb genes was codon-optimized for mammalian expression, and constructs were transiently expressed either in HEK 293 cells using FectoPRO® transfection reagent and protocol (Polyplus Transfection, New York, NY, USA) or into CHO cells using the ExpiCHO transient system (ThermoFisher Scientific) kit protocols. ExpiCHO expression followed protocols outlined by ThermoFisher for either mAb or CTLA4 Fc fusion transfection. For mAbs, plasmid containing gene sequence encoding for an IgG kappa light chain was co-transfected with a plasmid encoding for IgG heavy chain. For HEK293 expression, equal amounts by weight of heavy chain plasmid and kappa chain plasmid were co-transfected. For the Fc fusions, the single plasmid was transfected. Cells were allowed to grow for 7 days (HEK293) or 12 days (CHO) after which supernatants were collected for protein purification. CTLA4 Fc fusions and mAbs were screened for binding to protein A or protein G sensors via Octet QKe quantitation (Pall ForteBio Corp, Menlo Park, CA, USA). Expression was quantified on Octet with protein A or protein G sensors using standard curves, and mAbs/fusion proteins were purified with protein G or protein A/G affinity chromatography. For all protein constructs, Sodium Acetate pH 5.5 was used as binding and wash buffer, and elution was performed at pH 3.4. The purified proteins were neutralized and dialyzed into 20 mM Na acetate, pH 5.5, 140 mM NaCl for further analysis. The concentration of the mAbs and fusion proteins was measured via NanoDrop at 280 nm. Protein quality was assessed via analytical SEC and standard coomassie protein gels.

Table 2 lists the CTLA4 Fc fusions generated for three bovine IgG wildtype subclasses and mutations. Mutations were also made to the mAbs at identical positions as described in Table 2.

Additional mutations introduced to bovine IgGs but not made to the Fc fusions are: “DP” stabilization mutants for bIgG1a; Mutations to increase affinity to bFcRn; R433H on bIgG3a.

TABLE 2
Bovine Fc fusions with CTLA4.

| | Bovine IgG | |
|---|---|---|
| | subclass | |
| | Fc fusion | Residues involved |
| Fc Fusion name | partner | in mutation* |
| CTLA4 Fc bIgG1a | bIgG1a wildtype, | N/A |
| CTLA4 Fc bIgG1b | bIgG1b wildtype | |
| CTLA4 Fc bIgG1aWin | bIgG1a and | L234A_P235A_G237A |
| CTLA4 Fc bIgG1bWin | bIgG1b mutations | |
| CTLA4 Fc bIgG1aWinSS | for effector | L234A_P235A_G237A_A330S_P331S |
| CTLA4 Fc bIgG1bWinSS | function knockout | |
| CTLA4 Fc bIgG1aWinSAS | | L234A_P235A_G237A_P329S_P331S |
| CTLA4 Fc bIgG1bWinSAS | | |
| CTLA4 Fc bIgG1aSAS | | P329S_P331S |
| CTLA4 Fc bIgG1bSAS | | |
| CTLA4 Fc bIgG1aSAP | | P329S |
| CTLA4 Fc bIgG1bSAP | | |
| CTLA4bG2a | bIgG2a wildtype, | N/A |
| CTLA4bG2b | bIgG2b wildtype | |
| CTLA4bG2a_L432A | bIgG2a and | L432A |
| CTLA4bG2b_L432A | bIgG2b mutations | |
| CTLA4bG2a_N434A | for effector | N434A |
| CTLA4bG2b_N434A | function knockout | |
| CTLA4bG2a_M437A | | M437A |
| CTLA4bG2b_M437A | | |
| CTLA4bG2a_L432A_N434A | | L432A_N434A |
| CTLA4bG2b_L432A_N434A | | |
| CTLA4bG2a_L432A_M437A | | L432A_M437A |
| CTLA4bG2b_L432A_M437A | | |
| CTLA4bG2a_N434A_M437A | | N434A_M437A |
| CTLA4bG2b_L434A_M437A | | |
| CTLA4bG2a_L432A_N434A_M437A | | L432A_N434A_M437A |
| CTLA4bG2b_L432A_L434A_M437A | | |
| CTLA4 Fc bIgG3a | bIgG3a wildtype, | N/A |
| CTLA4 Fc bIgG3b | bIgG3b wildtype | |
| CTLA4 Fc bIgG3aWin | bIgG3a and | P234A_L235A_G237A |
| CTLA4 Fc bIgG3bWin | bIgG3b mutations | |
| CTLA4 Fc bIgG3aWinSAS | for effector | P234A_L235A_G237A_P329S_P331S |
| CTLA4 Fc bIgG3bWinSAS | function knockout | |
| CTLA4 Fc bIgG3aSAS | | P329S_P331S |
| CTLA4 Fc bIgG3bSAS | | |
| CTLA4 Fc bIgG1aSAP | | P329S |
| CTLA4 Fc bIgG3bSAP | | |

*Positions are numbered according to the Eu index as in Kabat (see FIG. 23)

Table 3 below describes mutations made to bIgG1b, IgG2b and IgG3b, including changes to amino acids resulting from nucleotide mutations. Table 3 also shows flanking sequence to the mutations. The same Fc mutations in Table 3 were made to bIgG1a, bIgG2a and IgG3a and the mutations result in identical amino acid sequences for the a and b allotypes.

TABLE 3
Fc Mutations made to the bovine IgG1b, IgG2b, and IgG3b.

| Subclass allotype mutation name: | Mutant Amino Acid and nucleotide |
|---|---|
| wildtype to mutated amino acids | sequences |
| bIgG1bWin (L234A_P235A_G237A): LPGG to | CDCCPPPEAAGAPSVFIFPP (SEQ ID NO.: 79) |
| AAGA | TGTGACTGCTGTCCACCTCCAGAGGCCGCCGG |
| | AGCCCCATCCGTGTTCATCTTTCCCCCT (SEQ |
| | ID NO.: 80) |
| bIgG1bWinSS (L234A_P235A_G237A; | CDCCPPPEAAGAPSVFIFPP (SEQ ID NO.: 79); |
| A330S_P331S): LPGG to AAGA; PAP to PSS | KVHNEGLPSSIVRTISRTK (SEQ ID NO.: 81) |
| | TGTGACTGCTGTCCACCTCCAGAGGCCGCCGG |
| | AGCCCCATCCGTGTTCATCTTTCCCCCT (SEQ |
| | ID NO.: 80); |
| | AAGGTGCATAACGAGGGCCTGCCATCCTCCAT |
| | CGTGAGAACAATCTCCCGCACCAAG (SEQ ID |
| | NO.: 82) |
| bIgG1bWinSAS (L234A_P235A_G237A; | CDCCPPPEAAGAPSVFIFPP (SEQ ID NO.: 79); |
| P329S_P331S): LPGG to AAGA; PAP to SAS | KVHNEGLSASIVRTISRTK (SEQ ID NO.: 83) |
| | TGTGACTGCTGTCCACCTCCAGAGGCCGCCGG |
| | AGCCCCATCCGTGTTCATCTTTCCCCCT (SEQ |
| | ID NO.: 80); |
| | AAGGTGCATAACGAGGGCCTGTCCGCTTCCAT |
| | CGTGAGAACAATCTCCCGCACCAAG (SEQ ID |
| | NO.: 84) |
| bigG1bSAS (P329S_P331S): PAP to SAS | KVHNEGLSASIVRTISRTK (SEQ ID NO.: 83) |
| | AAGGTGCATAACGAGGGCCTGTCCGCTTCCAT |
| | CGTGAGAACAATCTCCCGCACCAAG (SEQ ID |
| | NO.: 84) |
| bIgG1bSAP (P329S): PAP to SAP | KVHNEGLSAPIVRTISRTK (SEQ ID NO.: 85) |
| | AAGGTGCATAACGAGGGCCTGTCCGCTCCCAT |
| | CGTGAGAACAATCTCCCGCACCAAG (SEQ ID |
| | NO.: 86) |
| bIgG1b_DP1 (D216E): DP1 to EP1 | TKVDKAVEPTCKPSPCDCC (SEQ ID NO.: 87) |
| | ACAAAGGTGGACAAGGCCGTGGAGCCAACCT |
| | GCAAGCCAAGCCCCTGTGACTGCTGT (SEQ ID |
| | NO.: 88) |
| bIgG1b_DP2 (D270E): DP2 to EP2 | VVVDVGHDEPEVKFSWF (SEQ ID NO.: 89) |
| | GTGGTGGTGGATGTGGGCCACGACGAGCCTGA |
| | GGTGAAGTTCTCTTGGTTT (SEQ ID NO.: 90) |
| bIgG1b_DP1_DP2 (D216E_D270E): DP1 to | TKVDKAVEPTCKPSPCDCC (SEQ ID NO.: 87); |
| EP1_DP2 to EP2 | VVVDVGHDEPEVKFSWF (SEQ ID NO.: 89) |
| | ACAAAGGTGGACAAGGCCGTGGAGCCAACCT |
| | GCAAGCCAAGCCCCTGTGACTGCTGT (SEQ ID |
| | NO.: 88) |
| | GTGGTGGTGGATGTGGGCCACGACGAGCCTGA |
| | GGTGAAGTTCTCTTGGTTT (SEQ ID NO.: 90) |
| bIgG2b_L432A: LHNHYM to AHNHYM | VMHEAAHNHYMQKSTSK (SEQ ID NO.: 91) |
| | GTCATGCATGAGGCTGCCCACAATCATTATAT |
| | GCAGAAGAGCACATCTAAG (SEQ ID NO.: 92) |
| bIgG2b_N434A: LHNHYM to LHAHYM | VMHEALHAHYMQKSTSK (SEQ ID NO.: 93) |
| | GTCATGCATGAGGCTCTGCACGCCCATTATAT |
| | GCAGAAGAGCACATCTAAG (SEQ ID NO.: 94) |
| bIgG2b_M437A: LHNHYM to LHNHYA | VMHEALHNHYAQKSTSK (SEQ ID NO.: 95) |
| | GTCATGCATGAGGCTCTGCACAATCATTATGC |
| | CCAGAAGAGCACATCTAAG (SEQ ID NO.: 96) |
| bIgG2b_L432A_N434A: LHNHYM to AHNHYM; | VMHEAAHAHYMQKSTSK (SEQ ID NO.: 97) |
| LHNHYM to LHAHYM | GTCATGCATGAGGCTGCCCACGCCCATTATAT |
| | GCAGAAGAGCACATCTAAG (SEQ ID NO.: 98) |
| bIgG2b_L432A_M437A: LHNHYM to AHNHYM; | VMHEAAHNHYAQKSTSK (SEQ ID NO.: 99) |
| LHNHYM to LHNHYA | GTCATGCATGAGGCTGCCCACAATCATTATGC |
| | CCAGAAGAGCACATCTAAG (SEQ ID NO.: |
| | 100) |
| bIgG2b_N434A_M437A: LHNHYM to LHAHYM; | VMHEALHAHYAQKSTSK (SEQ ID NO.: 101) |
| LHNHYM to LHNHYA | GTCATGCATGAGGCTCTGCACGCCCATTATGC |
| | CCAGAAGAGCACATCTAAG (SEQ ID NO.: |
| | 102) |
| bIgG2b_L432A_N434A_M437A: LHNHYM to | VMHEAAHAHYAQKSTSK (SEQ ID NO.: 103) |
| AHNHYM; LHNHYM to LHAHYM; LHNHYM to | GTCATGCATGAGGCTGCCCACGCCCATTATGC |
| LHNHYA | CCAGAAGAGCACATCTAAG (SEQ ID NO.: |
| | 104) |
| bIgG3bWin (P234A_L235A_G237A): PLGG to | QCSKCPEAAGALSVFIFPP (SEQ ID NO.: 105) |
| AAGA | CAGTGTTCCAAGTGCCCAGAGGCCGCCGGAG |
| | CCCTGAGCGTGTTCATCTTTCCACCC (SEQ ID |
| | NO.: 106) |
| bigG3bWinSAS (P234A_L235A_G237A; | QCSKCPEAAGALSVFIFPP (SEQ ID NO.: 105); |
| P329S_P331S): PLGG to AAGA; PAP to SAS | KVNNKGLSASIVRTISRTK (SEQ ID NO.: 107) |
| | CAGTGTTCCAAGTGCCCAGAGGCCGCCGGAG |
| | CCCTGAGCGTGTTCATCTTTCCACCC (SEQ ID |
| | NO.: 106); |
| | AAGGTGAACAATAAGGGCCTGTCCGCCTCCAT |
| | CGTGAGAACAATCTCTCGCACCAAG (SEQ ID |
| | NO.: 108) |
| bIgG3bSAS (P329S_P331S): PAP to SAS | KVNNKGLSASIVRTISRTK (SEQ ID NO.: 107) |
| | AAGGTGAACAATAAGGGCCTGTCCGCCTCCAT |
| | CGTGAGAACAATCTCTCGCACCAAG (SEQ ID |
| | NO.: 108) |
| bIgG3bSAP (P329S): PAP to SAP | KVNNKGLSAPIVRTISRTK (SEQ ID NO.: 109) |
| | AAGGTGAACAATAAGGGCCTGTCCGCCCCAAT |
| | CGTGAGAACAATCTCTCGCACCAAG (SEQ ID |
| | NO.: 110) |
| bIgG3b_R433H: ALRNH to ALHNH | AVMHEALHNHYKEKSISR (SEQ ID NO.: 111) |
| | GCCGTGATGCACGAGGCTCTGCACAATCATTA |
| | CAAGGAGAAGAGCATCTCTCGC (SEQ ID NO.: |
| | 112) |

Bold and underlined: amino acid changes and corresponding mutated codons.
Nucleotide sequences translate to the amino acid sequence shown.

### Example 2

### bIgG Mutations Knock Out Effector Function

Several mutations were introduced to the Fc region of the bIgG1a allotype to knock out effector function: eliminate or reduce-to-negligible 1) the binding of bIgG1a to bFcgR1, bFcgR2, and bFcgR3, 2) the complement killing activity (CDC) in a cell-based assay, and 3) phagocytosis in an ADCP cell-based assay.

The “Winter” (or “Win”) site is just downstream from the hinge as previously reported for human IgG1. This LLGG “Winter” site for human IgG1 varies among species. For bovine IgG1 it is LPGG (FIG. 2). A mutation commonly referred to as “LALA” for human IgG1 is at Leu234Ala and Leu235Ala. For bovine IgG1, the corresponding Winter mutation is Leu234Ala, Pro235Ala, although an additional residue is also mutated. This additional mutation is Gly237Ala (numbered according to the Eu index as in Kabat).

The “PAP-to-SAS” (Pro329Ser_Pro331Ser) and “PAP-to-SAP” (Pro329Ser) mutations on bIgG1a were devised based on bIgG2, because bIgG2 does not trigger complement activity in a cell-based assay. Bovine IgG2a has a naturally occurring SAS site in CH2, while bIgG1a is PAP in this region as shown in FIG. 2. Thus, it was postulated that mutating PAP in bIgG1a to either SAS or SAP would eliminate CDC.

The “SS” mutation (Ala330Ser, Pro331Ser) on bIgG1a is based on a mutation to human IgG1 to resemble human IgG4 in this same region, PAP-to-PSS, because the hIgG4 subclass has weak affinity to FcgRs and thus negligible effector function. Indeed, the “SS” mutation on hIgG1 knocks out effector function. Bovine IgG1 has the same PAP sequence in this region as hIgG1, thus it was hypothesized that a PAP-to-PSS mutation on bIgG1 would knock out ADCC, ADCP, CDC and FcgR binding. The SS mutation was added to the Winter mutation on bIgG1.

The mutations described above were introduced on bIgG1a Fc: Winter mutation alone, L234A_P235A_G237A “Win” (SEQ ID NO. 16); Winter mutation plus SS mutation, L234A_P235A_G237A_A330S_P331S “WinSS” (SEQ ID NO. 18); Winter mutation plus SAS mutation, L234A_P235A_G237A_P329S_P331S “WinSAS” (SEQ ID NO. 17); SAS mutation alone, P329S_P331S “SAS” (SEQ ID NO. 15); and SAP mutation alone, P329S “SAP” (SEQ ID NO. 14). These mutations are aligned in FIG. 6.

The binding affinity of the above mutations for Fc gamma receptors was compared to bIgG1a wildtype. The mutants were also tested in functional cell-based assays and data indicate that both SAS and SAP mutations dramatically reduce CDC activity. SAS and SAP mutations also completely knock out ADCP. Unexpectedly, the “Winter” mutation appears to have enhanced rather than reduced complement activity in a cell-based CDC assay, and has negligible effects on ADCP in a cell-based assay. Also, unexpectedly, the “SS” mutation on bIgG1a doesn't fully knockout binding to bFcgR1 and bFcgR2. The SS mutation on bIgG1 has only slight reduction on CDC and only partially knocks down ADCP in cell-based assays.

### Example 3

### Effect of bIgG Fc Mutations on Bovine Fc Receptor Binding Affinities

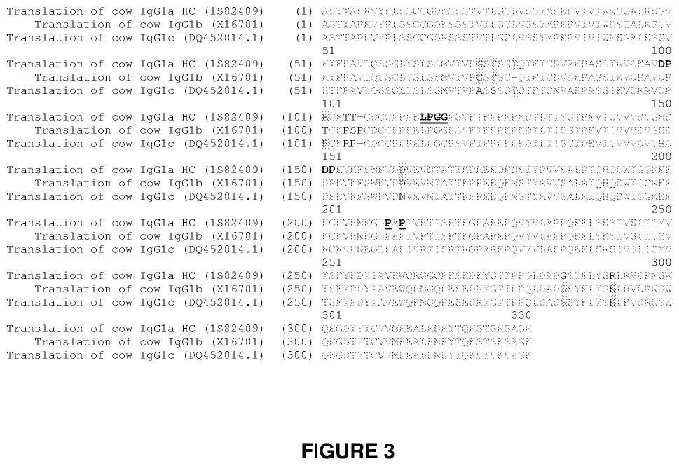

The ability to knock out binding to bovine Fc gamma receptors was evaluated by comparing SPR Biacore bFcgR affinity of bIgG1a and bIgG1b wildtype Fc to the bIgG1a and bIgG1b mutations, described above. Alignment of the constant region of the bIgG1a wildtype recombinant mAb with the five Fc mutants is shown in FIG. 6. Alignment of bIgG1a allotype with bIgG1b allotype is shown in FIG. 3. All mutations made to bIgG1 are identical for the a and b allotypes. Biacore methods for bFcgR1, R2, and R3 were performed as described in Example 1.

Table 4 shows the result of effector function Fc mutations on bIgG1a and bIgG1b binding to Fc gamma receptors. For bIgG1 wildtype, the two allotypes have similar binding affinities to bFcgR1, bFcgR2 and bFcgR3. While the Winter mutation (bIgG1aWin) knocked out binding to bFcgR3, it did not significantly affect bFcgR1 or bFcgR2 affinity. Adding the SS mutation to Winter (bIgG1aWinSS) somewhat reduced binding to bFcgR1 and bFcgR2 and retained negligible binding to bFcgR3. It is only with SAS added to Winter mutation (bIgG1aWinSAS), SAS mutation alone (bIgG1aSAS), or SAP mutation alone (bIgG1aSAP) that binding to all three Fc gamma receptors is knocked out. Bovine bFcgR1 had quite weak binding to IgG1aSAP and thus is effectively knocked out. With two exceptions, Table 4 shows that the bIgG1b wildtype and bIgG1b mutations had similar binding affinities to the bIgG1a wildtype and bIgG1a mutations, respectively. Thus, the two allotypes compare quite favorably except for 1) WinSS on bFcgR2 and 2) SAP on bFcgR1; however, the KDs for both of these mutants are significantly lower than for wildtype.

TABLE 4
Effect of bIgG1a and bIgG1b effector function Fc mutations
on binding to bovine Fc gamma receptors.

| CTLA4-bIgG1a | bFcgR1 | bFcgR2 | bFcgR3 |
|---|---|---|---|
| Fc fusions | KD (M) | KD (M) | KD (M) |
| bIgG1a wildtype | 1.20E−09 | 3.37E−09 | 1.43E−08 |
| bIgG1b wildtype | 4.63E−10 | 2.48E−09 | 1.37E−08 |
| bIgG1aWin | 7.45E−09 | 2.79E−09 | NBO |
| bIgG1bWin | 3.73E−09 | 3.24E−09 | LS |
| bIgG1aWinSS | 5.48E−08 | 5.25E−08 | LS |
| bIgG1bWinSS | 7.04E−08 | LS | LS |
| bIgG1aWinSAS | NBO | NBO | NBO |
| bIgG1bWinSAS | NBO | NBO | LS |
| bIgG1aSAS | NBO | NBO | NBO |
| bIgG1bSAS | NBO | NBO | LS |
| bIgG1aSAP | 1.48E−07 | NBO | NBO |
| bIgG1bSAP | NBO | NBO | LS |

NBO = No binding observed.
LS = low signal, indicating very weak binding.

### CDC Assay:

The CDC cell-based assay was developed and employed to characterize the effectiveness of the five CTLA4 bovine IgG1a Fc fusion proteins (FIG. 6) in mediating CDC, and to define key residues in the Fc region that determine CDC activity of the bovine IgG subtypes. The assay utilizes CHO target cells engineered to express canine CD80 which binds to CTLA4 on the Fc fusion proteins. These target cells have been used in past canine ADCC assays and were utilized in the CDC assay due to their dependability.

Incubation of the fusion protein-bound target cells with complement-preserved serum can result in Fc binding on the fusion proteins to complement component C1q initiating the complement cascade, ultimately forming membrane attack complexes. The pore-forming complexes mediate cell lysis of the target cells measured by loss of cell viability. If there is no Fc binding to C1q there is no resultant cell lysis/death.

Briefly, CD80-expressing CHO cells (target cells) were plated at 40,000 cells/well in CD CHO media in round-bottomed 96-well plates. Titrated fusion proteins in CD CHO media were added to the target cells and allowed to bind for 60 minutes at 37° C. Bovine complement preserved serum (30% in CD CHO media) was added to the plates for 45 minutes at 37° C. Cell viability was then measured using CellTiter-Glo and data were expressed as “cell viability % of control” calculated using no fusion protein+complement preserved serum controls.

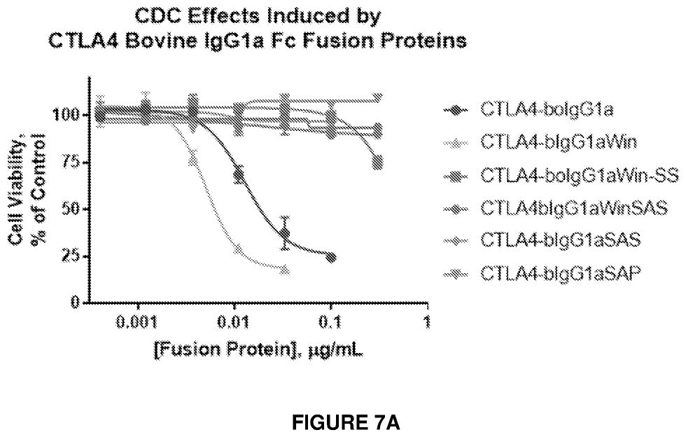

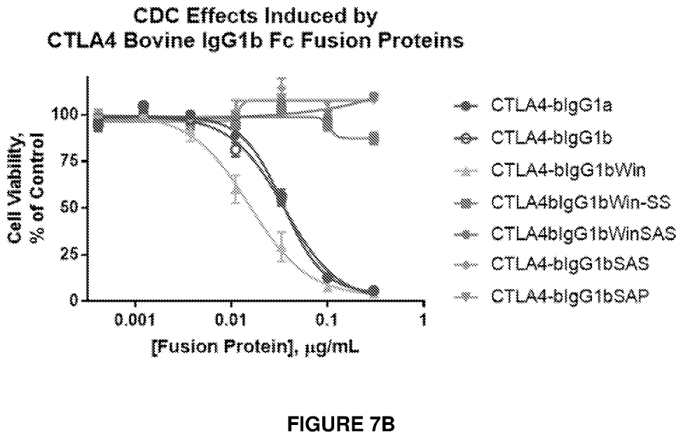

As shown in FIG. 7 and Table 5, the Win mutation alone does not knockout CDC activity of bIgG1a or bIgG1b Fc. It appears to potentiate the CDC activity. The WinSS mutation triggers CDC, although quite weakly. WinSAS, SAS alone, and SAP mutations greatly reduce or completely knockout CDC activity for both bIgG1a Fc and bIgG1b Fc.

### In Silico Modeling

The FAb sequence of a bovine antibody was tethered to the sequences of Fc domains of various bovine backbone subclasses using their respective hinge regions. The antibody homology modeling feature of Molecular Operating Environment 2019.0102 program (MOE2019.0102) developed by Chemical Computing Group was implemented to model the 3D structure of each of wild-type (WT) and mutant constructs of each bovine backbone allotype. MOE2019.0102 provides a flexible and automated graphical user interface for antibody homology modeling and the default parameters were selected for the modeling runs.

To model the mAb structures, a knowledge-based approach was employed using the built-in PDB database of MOE2019.0102. The sequence-to-profile alignment algorithm uses a scoring algorithm to rank the heavy and light chain sequence templates and scores higher than 85% ensure the selection of antibody templates with physically realistic structures. Once the CDR and loop templates were joined, the model was then optimized using the same pipeline. The structural stability of the models was verified using Ramachandran Plots, which checks the stereochemical quality of a protein structure.

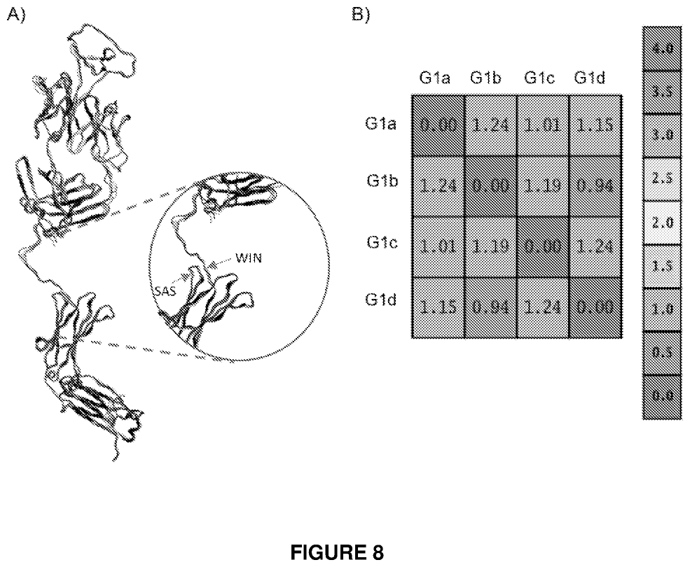

In order to understand the structure-activity relationship across the mutated constructs, the method described above was performed on bIgG1a_WINSAS, bIgG1b_WINSAS, bIgG1c_WINSAS and bIgG1d_WINSAS. The structures were overlayed, and the mutated residues are indicated by arrows (FIG. 8A). An RMSD plot was generated to calculate the root mean square deviation of the four structures relative to each other (FIG. 8B). An RMSD value of 2.0 Å or lower is considered the standard for considering two structures to be alike. Results indicated that bIgG1a_WINSAS construct was identical to its other allotype counterpart, bIgG1b WINSAS since their RMSD was 1.24 Å. This was also supported in the CDC assay (FIG. 7) where bIgG1a_WINSAS showed the same loss of CDC activity as bIgG1b_WINSAS. This offers more support to show that the antibody models that were generated by the method described above provides an accurate representation of the bovine backbone structures. Further, comparing the antibody models of bIgG1a_WINSAS and bIgG1b_WINSAS to bIgG1c_WINSAS and bIgG1d_WINSAS, the RMSD values ranged between 0.94-1.24 Å and thus we can predict with a high degree of certainty that bIgG1c_WINSAS and bIgG1d_WINSAS exhibit the same lack of functional activity (CDC, ADCP and ADCC) as bIgG1a_WINSAS.

### ADCP Assay:

The ADCP cell-based assay was developed and employed to characterize the effectiveness of the five CTLA4 bovine IgG1a Fc fusion proteins (FIG. 6) in mediating ADCP, and to define key residues in the Fc region that determine ADCP activity of the bovine IgG subtypes. The assay utilizes CHO target cells engineered to express canine CD80, which binds to canine CTLA4 on the Fc fusion proteins. These target cells have been used in past canine ADCC assays and were utilized in the CDC assay due to their dependability.

For this assay, incubation of the Fc fusion protein/target cell complexes with bovine alveolar macrophages can result in Fc binding to Fc gamma receptors on the macrophages, bridging the CHO target cells and the macrophage effectors and thereby initiating phagocytosis of the target cells. ADCP is measured by signal intensity and frequency of a pH-sensitive fluorescent dye within the population of effector macrophages in the co-culture, wherein fluorescent cells are indicative of an effector that has successfully internalized a target cell into the acidic lysosome.

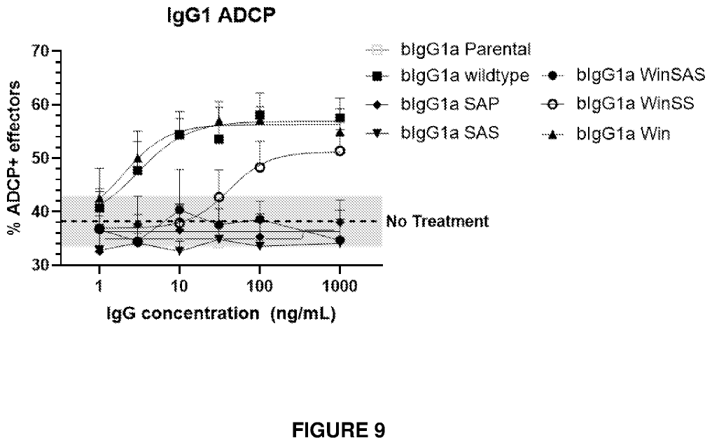

Briefly, canine CD80-expressing CHO cells (CD80 target cells) or parental CHO cells not expressing CD80 (parental target cells) were pre-stained with pHrodo red dye for 30 minutes at 37 degrees C. Cells were then incubated with CTLA4-Fc fusion proteins for 20 minutes to mediate CTLA4:CD80 binding, and subsequently co-cultured with pre-plated adherent bovine alveolar macrophages stained with a cell marker (CellTrace Violet, CTV) to aid in later identification. 60,000 target cells were plated with 30,000 effector macrophages in a 96 well plate. Cells were then co-cultured for 6 hours, and subsequently removed from plates and analyzed by flow cytometry to identify effector cells (CTV+) that have successfully performed ADCP (pHrodo+). As shown in FIG. 9 and Table 5, wildtype bIgG1a is a potent activator of ADCP in bovine alveolar macrophages. The bIgG1aWin mutation alone does not significantly affect ADCP activity of bIgG1a Fc, and may slightly potentiate ADCP, as seen for CDC activity. The WinSS mutation partially knocks down ADCP, while the WinSAS, SAS alone and SAP mutations on bIgG1a completely abrogate ADCP function.

Incubation of the wildtype bIgG1a with target cells not expressing canine CD80 (IgG1 parental) also does not trigger ADCP, indicating the observed target cell internalization requires the specific activity of the fusion protein and is not mediated by alternative mechanisms.

Phagocytosis (ADCP) is mediated by multiple human Fc gamma receptors including FcgR1 and DNA sequence analysis indicates motifs of the IgG binding domain of the bovine FcgR1 are highly conserved compared to its human and mouse counterparts. The cell based ADCP data in FIG. 9 and Table 5 compare favorably to the binding affinities of bIgG1a and mutants to bFcgR1 in Table 4. The Winter mutation (bIgG1aWin) did not significantly affect bFcgR1 affinity. Adding the SS mutation to Winter (bIgG1aWinSS) knocked down binding to bFcgR1 somewhat but did not completely knock out binding. It is only with SAS added to Winter mutation (bIgG1aWinSAS), SAS mutation alone (bIgG1aSAS), or SAP mutation alone (bIgG1aSAP) that binding to bFcgR1 is knocked out. Bovine bFcgR1 had only negligible binding to IgG1aSAP and thus is effectively knocked out.

TABLE 5
CDC and ADCP effects induced by bovine IgG1a
and IgG1b Fc wildtype and mutations.

| | CDC | ADCP |
|---|---|---|
| CTLA4-bIgG1a Fusions | EC50 Value | EC50 Value |
| CTLA4 Fc bIgG1a wildtype | 0.013 μg/mL | 0.003 μg/mL |
| CTLA4 Fc bIgG1aWin | 0.006 μg/mL | 0.002 μg/mL |
| CTLA4 Fc bIgG1aWin-SS | Partial CDC | 0.041 μg/mL |
| CTLA4 Fc bIgG1aWin-SAS | No CDC | No ADCP |
| CTLA4 Fc bIgG1aSAS | No CDC | No ADCP |
| CTLA4 Fc bIgG1aSAP | No CDC | No ADCP |

| | CDC |
|---|---|
| CTLA4-bIgG1b Fusions | EC50 Value |
| CTLA4 Fc bIgG1a wildtype (control) | 0.034 μg/mL |
| CTLA4 Fc bIgG1b wildtype | 0.036 μg/mL |
| CTLA4 Fc bIgG1bWin | 0.015 μg/mL |
| CTLA4 Fc bIgG1bWin-SS | Partial CDC |
| CTLA4 Fc bIgG1bWin-SAS | No CDC |
| CTLA4 Fc bIgG1bSAS | No CDC |
| CTLA4 Fc bIgG1bSAP | No CDC |

### Example 4

### bIgG DP Mutations for Stabilization

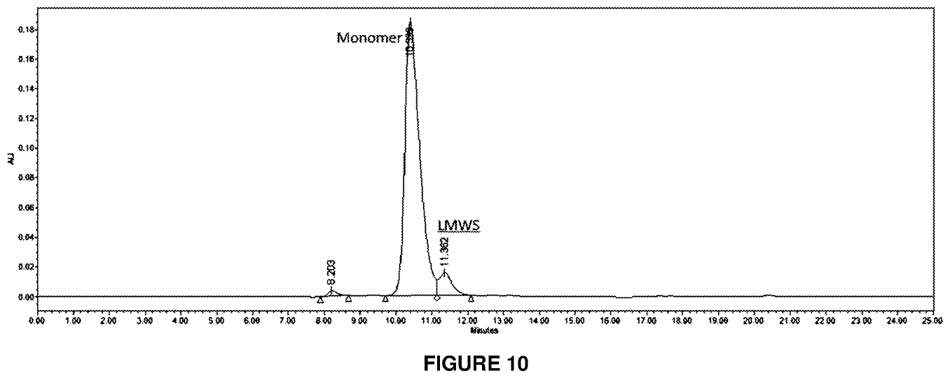

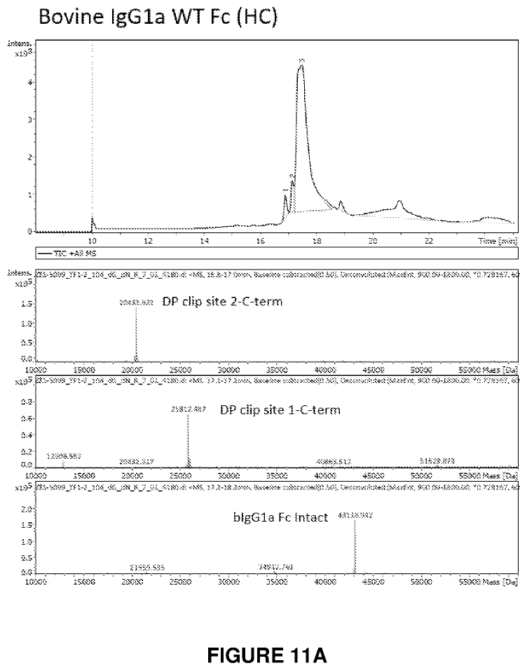

On analytical investigation of the bovine IgG1a native wild-type subclass, lower molecular weight species (LMWS) were observed upon analysis by Size Exclusion Chromatography (SEC), as shown in FIG. 10. To investigate the possible cause, the sample was subjected to Mass Spectral analysis, which identified two clipped sites located in the bovine IgG1a constant heavy chain between D216 and P217 and between D270 and P271. These two sites of chemical cleavage are contained in the native amino acid sequence of the bovine IgG1a Fe (NCBI reference 1S82409). Elimination of cleavage/clipping sites in the constant domain of bIgG1a is desirable to increase the conformational stability, intact monomer percentage, and overall developability of the bovine IgG1 subclass. Mutations were made to the constant domain of the bovine IgG1a to eliminate these cleavage/clipping sites (Table 6). These identified cleavage sites are dependent on amino acid sequence, which can trigger the non-enzymatic breakage of the bond between Asp (D) and Pro (P) amino acids in the IgG protein. While intentional cleavage of the DP bond is well documented in the literature using acid and heat, not all DP sites are conformationally accessible and do not cleave under short exposure to acid, even at elevated temperatures. Therefore, it is not evident if these sequence site liabilities would automatically lead to clipping of the bIgG1a Fc.

TABLE 6
Mutations of the DP sites in the bovine IgG1a constant heavy chain

| Identification | Mutation Made |
|---|---|
| Single mutation DP site 1 | D216E |
| Single mutation DP site 2 | D270E |
| Double mutation DP site 1 & 2 | D216E and D270E |

Mutations of DP-to-EP described above were constructed via quick change mutagenesis of the mammalian expression vector containing the bIgG1a. The monoclonal antibody (mAbs) mutants were expressed in mammalian suspension cell systems, EXPICHO-S (Chinese Hamster Ovary) cells, obtained from Thermo Fisher. Suspension EXPICHO-S cells were maintained in EXPICHO expression medium (Gibco) between 0.14 and 8.0×10e6 cells/ml. Cells are diluted following the ExpiCHO Protocol user manual on Day −1 and transfection day. Diluted cells are transfected as described in the protocol using reagents sourced from ExpiFectamine CHO Transfection Kit (Gibco) following Max Titer conditions. Following 12-14 days of incubation, the cultures are harvested and clarified. Conditioned media was loaded onto MabSelect Sure LX (GE Healthcare) which had been pre-equilibrated with PBS. Following sample load, the resin was washed with PBS and then with 20 mM sodium acetate, pH 5.5. Samples were eluted from the column with 20 mM acetic acid, pH 3.5. Following elution, pools were made and neutralized with the addition of 1 M sodium acetate to 4%. Depending on available volume and intended use, samples were sometimes exchanged into a final buffer (e.g. PBS, other). Concentration was measured by absorbance at 280 nm.

Analytical SEC was conducted using a TSK gel Super SW3000, 4.6 mm, 10×30 cm, 4 m column from TOSOH BioScience, in 200 mM NaPhosphate pH 7.2 running buffer at 0.25 ml/minute.

Mass spectrometric (mass spec) analysis was performed on the wild-type native bovine IgG1a and mutants. The samples were deglycosylated using PNGase F (New England Biolabs) and reduced with DTT (Thermo). Samples were analyzed using a maXis plus ESI instrument (Bruker). FIG. 10 confirms that the mass of the identified DP clip sites are evident in the native bIgG1a heavy chain. The bIgG1a double mutation D216E (site 1) and D270E (site 2) was also analyzed by mass spec in the same manner as the native Fc. It is evident from this data that the DP clip is no longer present.

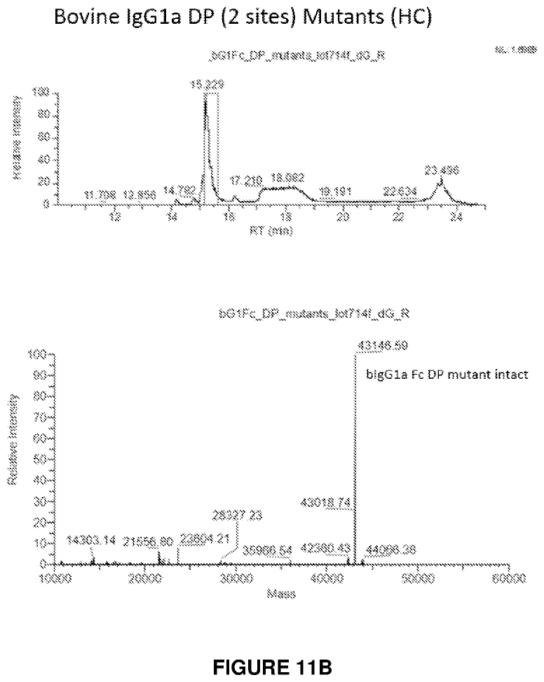

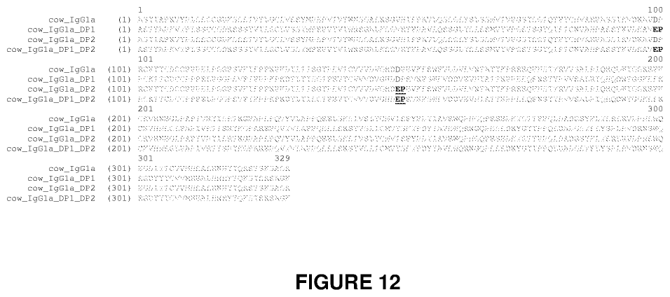

The D-P to E-P mutations introduced (FIG. 12) removed the cleavage/clips sites from the bIgG1a Fc as evidenced by mass spectral analysis (FIG. 11). The removal of these cleavage sites can improve the homogeneity, intact IgG purity and overall developability of this subclass for therapeutic uses.

### In Silico Modeling

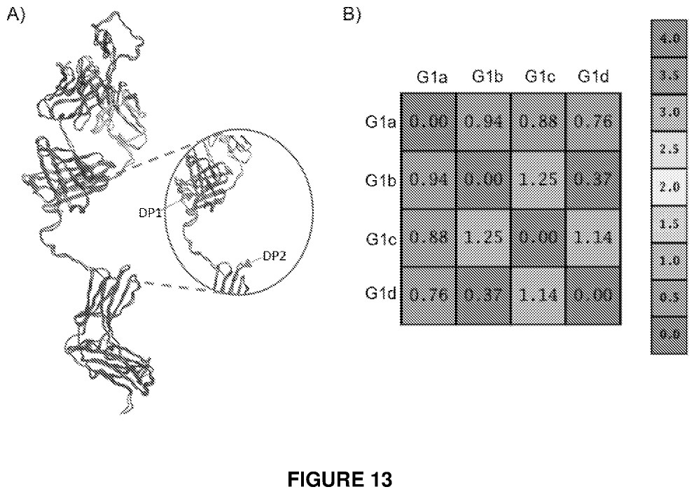

The method described previously for bIgG1a_WINSAS, bIgG1b_WINSAS, bIgG1c_WINSAS and bIgG1d_WINSAS was performed on bIgG1DP1_DP2 allotypes. Results shown in FIG. 13 indicated that bov_G1a_DP1_DP2 construct was identical to its other allotype counterparts, bIgG1b_DP1_DP2, bIgG1c_DP1_DP2 and bIgG1d_DP1_DP2 with RMSD values ranging between 0.76-1.14 Å. Therefore, based on both experimental and model results, we predict with a high degree of certainty that DP1_DP2 mutants in allotypes bIgG1b, bIgG1c and bIgG1d will behave in a similar fashion to bIgG1a_DP1_DP2.

### Example 5

### bIgG Mutations Knock Out Effector Function

Several mutations were introduced to the Fc region of the bIgG2a allotype to knock out effector function: eliminate or reduce-to-negligible 1) the binding of bIgG2a to the only Fc gamma receptor it engages, bFcg2R, and 2) phagocytosis in an ADCP cell-based assay.

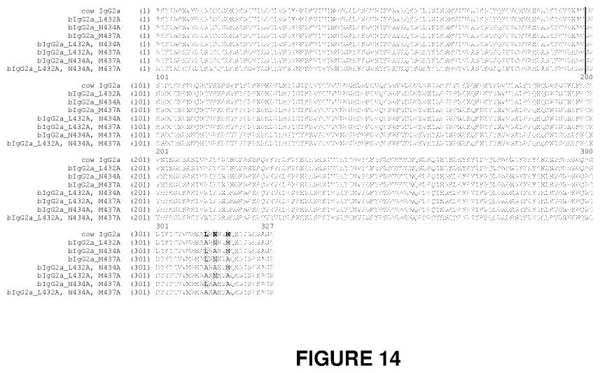

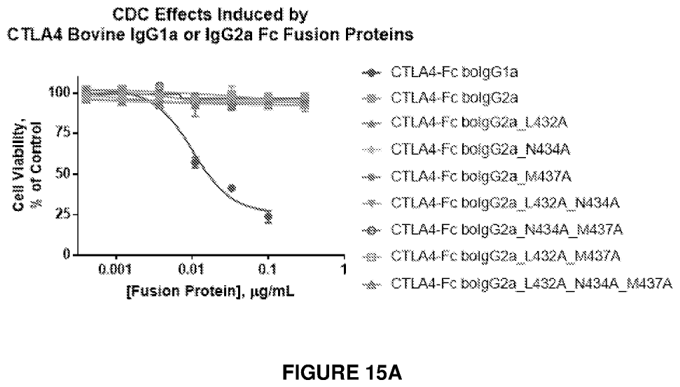

HDX epitope mapping of bovine IgG2a to bFcg2R indicated a discontinuous epitope on bIgG2a. In a campaign to eliminate or reduce-to-negligible the binding to bFcg2R, an alanine mutation panel in this region of bIgG2a was created. The mutations that were effective at dramatically knocking down Fc gamma receptor binding while retaining neonatal Fc receptor affinity are illustrated in FIG. 14. These mutations are L432A, N434A, and M437A. In addition, the following combinations of L432A, N434A, and M437A successfully knocked out binding to bFcg2R: L432A_N434A; L432A_M437A; N434A_M437A; and L432A_N434A_M437A.

### Example 6

### Effect of bIgG Alanine Mutations on bFcg2R and bFcRn Binding Affinities

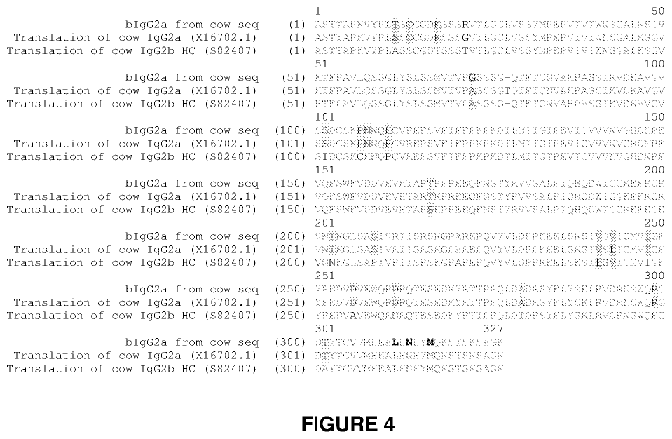

The ability to knock out binding to bFcg2R while retaining affinity to bFcRn was evaluated by comparing SPR Biacore affinity differences between the bIgG2a wildtype Fc and bIgG2a alanine mutations described above. Alignment of the constant region of the bIgG2a wildtype (wt) recombinant mAb with the seven Fc mutants is shown in FIG. 14. Alignment of bIgG2a allotype with bIgG2b allotype is shown in FIG. 4. All mutations made to bIgG2 are identical for the a and b allotypes. The bIgG2a wt and mutants were produced as mAbs and run on Biacore for binding affinities to the bovine Fc gamma receptors and bovine neonatal Fc receptor. Biacore methods for bFcg2R, bFcRn, bFcgR1, bFcgR2, and bFcgR3 were performed as described in the Example sections above.

As shown in Table 7, the three single point mutations on bIgG2a, L432A, N434A, and M437A dramatically knock down binding to bFcg2R, resulting in nearly total knockout of binding. The double mutation L432A_N434A reduces binding to bFcg2R by 4-fold compared to wildtype binding. The double mutations L432A_M437A; N434A_M437A and the triple mutation L432A_N434A_M437A completely knock out binding to Fcg2R when compared to the wildtype Fc.

The bovine Fcg2R knockout mutations had a varied effect on binding to bovine FcRn. Recycling of IgG through the pH-dependent FcRn requires strong affinity at pH6 to avoid endosomal degradation and weak affinity at pH7.4 to release the rescued IgG back into circulation. The three single-point mutations had the least effect on bFcRn binding at pH6, retaining strong affinity similar to wildtype, while the double mutations and the triple mutation had the most effect on bFcRn affinity at pH 7.4, reducing affinity below detection limit.

TABLE 7
Binding affinity of bIgG2a wt and alanine mutant mAbs to bFcg2R and bFcRn.

| | bFcg2R | Fcg2R KD reduction | bFcRn, pH 6 | bFcRn, pH 7.4 |
|---|---|---|---|---|
| bIgG2a mAbs | KD (M) | from WT | KD (M) | KD (M) |
| bIgG2a wildtype | 1.33E−08 | N/A | 2.54E−10 | 1.47E−07 |
| bIgG2a_L432A | LS | Near total knockout | 5.79E−10 | LS |
| bIgG2a_N434A | LS | Near total knockout | 1.04E−09 | LS |
| bIgG2a_M437A | LS | Near total knockout | 5.90E−10 | LS |
| bIgG2a_L432A_N434A | 5.93E−08 | 4-fold reduction | 1.70E−09 | LS |
| bIgG2a_L432A_M437A | NBO | total knockout | 3.03E−09 | NBO |
| bIgG2a_N434A_M437A | NBO | total knockout | 2.31E−09 | NBO |
| bIgG2a_L432A_N434A_M437A | NBO | total knockout | 6.96E−09 | NBO |

NBO = No binding observed.
LS = low signal, indicating very weak binding.

The bIgG2a wildtype and all bIgG2a Fcg2R knockout mutations had no or negligible binding to bFcgR1, bFcgR2 and bFcgR3 as shown in Table 8.

TABLE 8
Binding affinity of bIgG2a wt and alanine
mutants to bFcgR1, bFcgR2, and bFcgR3.

| | bFcgR1 | bFcgR2 | bFcgR3 |
|---|---|---|---|
| bIgG2a mAbs | KD (M) | KD (M) | KD (M) |
| bIgG2a wildtype | NBO | NBO | NBO |
| bIgG2a_L432A | NBO | NBO | NBO |
| bIgG2a_N434A | NBO | NBO | NBO |
| bIgG2a_M437A | NBO | NBO | NBO |
| bIgG2a_L432A_N434A | NBO | NBO | NBO |
| bIgG2a_L432A_M437A | NBO | NBO | NBO |
| bIgG2a_N434A_M437A | NBO | NBO | NBO |
| bIgG2a_L432A_N434A_M437A | NBO | NBO | NBO |

NBO = No binding observed.

In addition to mAb reagents for bIgG2a wildtypes and mutants shown in Table 7 and 8, CTLA4-Fc fusion versions for both bIgG2 a and b allotypes were prepared as described above. Examination of bFcg2R affinity with wildtype bIgG2a allotype CTLA4-Fc fusion compared with wildtype bIgG2b allotype CTLA4-Fc fusion shows nearly identical KD, 4.17E-08 vs. 3.14E-08 (Table 9). Comparing the two allotypes for all of the Fc mutations also shows very similar bFcg2R binding affinities.

TABLE 9
Comparison of bIgG2a wt and alanine mutant Fc fusions
vs. bIgG2b wt and alanine mutant Fc fusions to bFcg2R.

| | bFcg2R |
|---|---|
| CTLA4 fusions | KD (M) |
| bIgG2a wildtype | 4.17E−08 |
| bIgG2b wildtype | 3.14E−08 |
| bIgG2a_L432A | NBO |
| bIgG2b_L432A | LS |
| bIgG2a_N434A | NBO |
| bIgG2b_N434A | LS |
| bIgG2a_M437A | WB |
| bIgG2b_M437A | LS |
| bIgG2a_L432A_N434A | NBO |
| bIgG2b_L432A_N434A | NBO |
| bIgG2a_L432A_M437A | NBO |
| bIgG2b_L432A_M437A | NBO |
| bIgG2a_N434A_M437A | NBO |
| bIgG2b_N434A_M437A | NBO |
| bIgG2a_L432A_N434A_M437A | NBO |
| bIgG2b_L432A_N434A_M437A | NBO |

NBO = No binding observed.
WB = weak binding.
LS = low signal, indicating very weak binding.

### CDC Assay:

The CDC cell-based assay was developed and employed to characterize the effectiveness of the seven CTLA4 bovine IgG2a Fc fusion proteins (FIG. 14) in mediating CDC, and to define key residues in the Fc region that determine CDC activity of the bovine IgG subtypes. The assay utilizes CHO target cells engineered to express canine CD80 which binds to CTLA4 on the Fc fusion proteins. These target cells have been used in past canine ADCC assays and were utilized in the CDC assay due to their dependability. The assay method was performed as described for bIgG2a CTLA4 Fc fusions above.

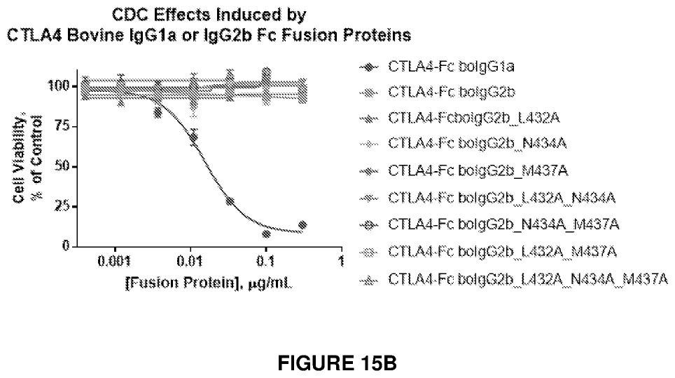

As shown in FIG. 15 and Table 10, neither of the two bovine IgG2 wildtype allotypes, bIgG2a and bIgG2b, trigger CDC in a cell-based assay, while the positive control bIgG1a has similar CDC activity as previous runs. In addition, none of the seven alanine mutations alter bIgG1a CDC function.

### In Silico Modeling:

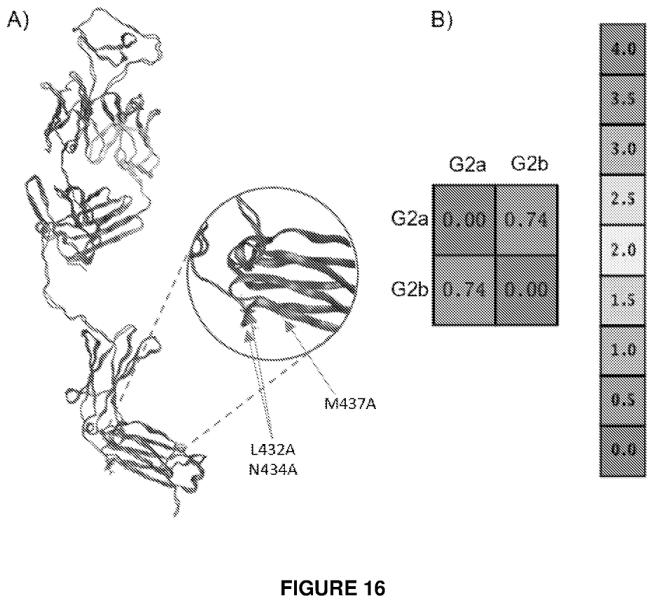

The method described previously for bIgG1a_WINSAS, bIgG1b_WINSAS, bIgG1c_WINSAS and bIgG1d_WINSAS was performed on bIgG2a mutants. No changes in RMSD over 2A were observed for this subclass, indicating that the two allotypes fold similarly (FIG. 16B). As with the previous subclass, we rationalized that if having all possible mutations in single construct (L432, N434A and M437A) did not make a significant change in protein fold, individual mutations should not affect the protein model either. These models are also supported by the effector function data observed using CDC assay. These antibody models taken together with the SPR binding data (i.e., experimental data) in Table 7 indicate that bIgG2a_L432A_N434A_M437A, bIgG2b_L432A_N434A_M437A and various combinations of these mutations (listed in Tables 2 and 7) fold and bind in an identical manner and hence would have similar lack of ADCP and ADCC effector functions.

### ADCP Assay:

The ADCP cell-based assay was developed and employed to characterize the effectiveness of the seven CTLA4 bovine IgG2a Fc fusion proteins (FIG. 14) in mediating ADCP, and to define key residues in the Fc region that determine ADCP activity of the bovine IgG subtypes. The assay utilizes CHO target cells engineered to express canine CD80 which binds to CTLA4 on the Fc fusion proteins. These target cells have been used in past canine ADCC assays and were utilized in the CDC assay due to their dependability.

For this assay, incubation of the Fc fusion protein/target cell complexes with bovine alveolar macrophages can result in Fc binding to Fc gamma receptors on the macrophages, bridging the CHO target cells and the macrophage effectors and thereby initiating phagocytosis of the target cells. ADCP is measured by signal intensity and frequency of a pH-sensitive fluorescent dye within the population of effector macrophages in the co-culture, wherein fluorescent cells are indicative of an effector that has successfully internalized a target cell into the acidic lysosome.

Briefly, canine CD80-expressing CHO cells (CD80 target cells) or parental CHO cells not expressing CD80 (parental target cells) are pre-stained with pHrodo red dye for 30 minutes at 37 degrees C. Cells are then incubated with CTLA4-Fc fusion proteins for 20 minutes to mediate CTLA4:CD80 binding, and subsequently co-cultured with pre-plated adherent bovine alveolar macrophages stained with a cell marker (CellTrace Violet, CTV) to aid in later identification. 60,000 target cells are plated with 30,000 effector macrophages in a 96 well plate. Cells are co-cultured for 6 hours, and subsequently removed from plates and analyzed by flow cytometry to identify effector cells (CTV+) that have successfully performed ADCP (pHrodo+).

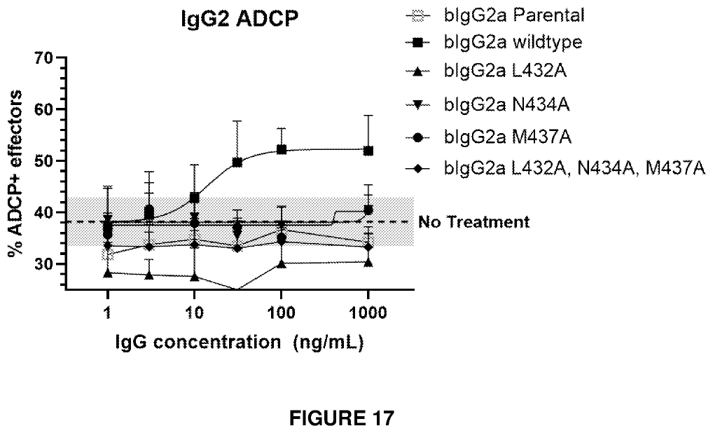

As shown in FIG. 17 and Table 10, while the bovine IgG2 subclass is not as potent at activating ADCP in bovine alveolar macrophages as the bovine IgG1 subclass (see FIG. 9 and Table 5), bIgG2 is capable of triggering ADCP at higher concentrations. All bIgG2a alanine mutations eliminate the ADCP activity of wildtype bIgG2a.

TABLE 10
CDC and ADCP effects induced by bovine IgG2a
and IgG2b Fc wildtype and mutations.

| | CDC | ADCP |
|---|---|---|
| CTLA4-bIgG2a Fusions | EC50 Value | EC50 Value |
| CTLA4 Fc bIgG1a wildtype (control) | 0.010 μg/mL | 0.003 μg/mL |
| CTLA4 Fc bIgG2a wildtype | No CDC | 0.014 μg/mL |
| CTLA4 Fc bIgG2a_L432A | No CDC | No ADCP |
| CTLA4 Fc bIgG2a_N434A | No CDC | No ADCP |
| CTLA4 Fc bIgG2a_M437A | No CDC | No ADCP |
| CTLA4 Fc bIgG2a_L432A_N434A | No CDC | No ADCP |
| CTLA4 Fc bIgG2a_L432A_M437A | No CDC | |
| CTLA4 Fc bIgG2a_N434A_M437A | No CDC | |
| CTLA4 Fc bIgG2a_L432A_N434A_M437A | No CDC | |

| | CDC |
|---|---|
| CTLA4-bIgG2b Fusions | EC50 Value |
| CTLA4 Fc bIgG1a wildtype (control) | 0.015 μg/mL |
| CTLA4 Fc bIgG2b wildtype | No CDC |
| CTLA4 Fc bIgG2b_L432A | No CDC |
| CTLA4 Fc bIgG2b_N434A | No CDC |
| CTLA4 Fc bIgG2b_M437A | No CDC |
| CTLA4 Fc bIgG2b_L432A_N434A | No CDC |
| CTLA4 Fc bIgG2b_L432A_M437A | No CDC |
| CTLA4 Fc bIgG2b_N434A_M437A | No CDC |
| CTLA4 Fc bIgG2b_L432A_N434A_M437A | No CDC |

### Example 7

### bIgG Mutations Knockout Effector Function

Several mutations were introduced to the Fc region of the bIgG3a allotype to knockout effector function: eliminate or reduce-to-negligible 1) the binding of bIgG3a to bFcgR1, bFcgR2, and bFcgR3, 2) the complement killing activity (CDC) in a cell-based assay, and 3) phagocytosis in an ADCP cell-based assay.

The “Winter” (or “Win”) site is just downstream from the hinge as described above for human IgG1. This LLGG “Winter” site for human IgG1 varies among species. For bovine IgG3 it is PLGG (FIG. 2). As discussed above, a mutation commonly referred to as “LALA” for human IgG1 is at Leu234Ala and Leu235Ala.

For bIgG3, the corresponding Winter mutation is Pro234Ala, Leu235Ala, although an additional residue is also mutated. This additional mutation is Gly237Ala (numbered according to the Eu index as in Kabat).

The “PAP-to-SAS” (Pro329Ser, Pro331Ser) and “PAP-to-SAP” (Pro329Ser) mutations on bIgG3a were devised based on bIgG2. Bovine IgG2 does not trigger complement activity in a cell-based assay. Bovine IgG2a has a naturally occurring SAS site in CH2, while bIgG3a is PAP in this region as shown in FIG. 2. Thus, it was postulated that mutating PAP in bIgG3a to either SAS or SAP would eliminate CDC.

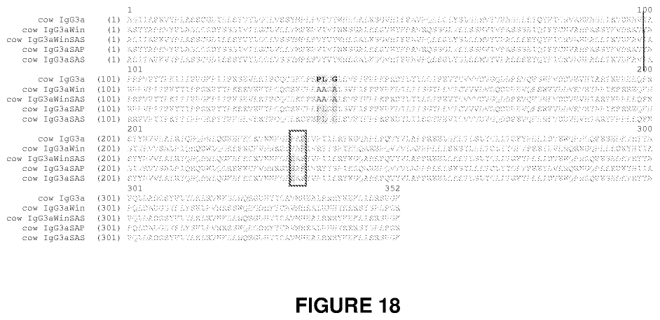

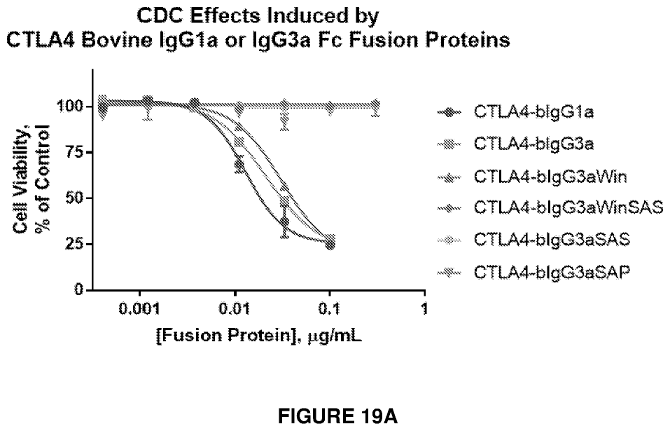

The mutations described above were introduced on bIgG3a Fc: Winter mutation alone, P234A_L235A_G237A “Win” (SEQ ID NO. 29); Winter mutation plus SAS mutation, P234A_L235A_G237A_P329S_P331S “WinSAS” (SEQ ID NO. 30); SAS mutation alone, P329S_P331S “SAS” (SEQ ID NO. 28); and SAP mutation alone, P329S “SAP” (SEQ ID NO. 27). These mutations are aligned in FIG. 18 and numbered according to the Eu index as in Kabat.

The binding affinity of the above mutations for Fc gamma receptors was compared to bIgG3a wildtype. The mutants were also tested in functional cell-based assays and data indicate that while the Win mutation alone slightly weakened CDC, the WinSAS, SAS-only and SAP-only mutations completed knocked out CDC activity. All four Win, WinSAS, SAS and SAP mutations completely knock out ADCP function.

### Example 8

### Effect of bIgG Fc Mutations on Bovine Fc Receptor Binding Affinities

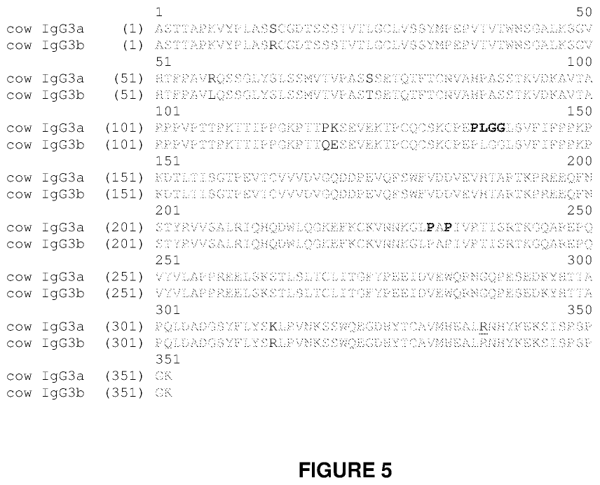

The ability to knock out binding to bovine Fc gamma receptors was evaluated by comparing SPR Biacore bFcgR affinity of bIgG3a and bIgG3b wildtype Fc to the bIgG3a and bIgG3b mutations described above. Alignment of the constant region of the bIgG3a wildtype recombinant mAb with the four Fc mutants is shown in FIG. 18. Alignment of bIgG3a allotype with bIgG3b allotype is shown in FIG. 5. All mutations made to bIgG3 are identical for the a and b allotypes

Biacore methods for bFcgR1, R2, and R3 were performed as described in Examples above. Table 11 shows the result of effector function bIgG3a and bIgG3b Fc mutations on binding to Fe gamma receptors. For bIgG3 wildtype, the two allotypes have similar binding affinity to bFcgR1, 9.36E-09 compared to 1.25E-08. Although the two allotypes have different KDs for bFcgR2 and bFcgR3, both wildtype allotypes do bind. All of the mutations, bIgG3Win, bIgG3WinSAS, bIgG3SAS and bIgG3SAP for both the a and b allotypes knocked down binding to bFcgR1, bFcgR2 and bFcgR3 to negligible affinities. Thus, for knocking out receptor binding, the two allotypes compare similarly.

TABLE 11
Binding affinity of bIgG3a and bIgG3b wildtype and mutant
CTLA4-Fc fusions to bovine Fc gamma receptors.

| CTLA4-bIgG3a | bFcgR1 | bFcgR2 | bFcgR3 |
|---|---|---|---|
| Fc fusions | KD (M) | KD (M) | KD (M) |
| bIgG3a wildtype | 9.36E−09 | 6.89E−08 | 7.17E−06 |
| bIgG3b wildtype | 1.25E−08 | 7.06E−07 | 4.96E−08 |
| bIgG3aWin | LS | NBO | NBO |
| bIgG3bWin | NBO | NBO | LS |
| bIgG3aWinSAS | NBO | NBO | WB |
| bIgG3bWinSAS | NBO | NBO | LS |
| bIgG3aSAS | NBO | NBO | WB |
| bIgG3bSAS | NBO | NBO | LS |
| bIgG3aSAP | NBO | NBO | LS |
| bIgG3bSAP | NBO | NBO | LS |

NBO = No binding observed.
WB = weak binding.
LS = low signal, indicating very weak binding.

### CDC Assay:

The CDC cell-based assay was developed and employed to characterize the effectiveness of the four CTLA4 bovine IgG3a Fc fusion proteins (FIG. 18) in mediating CDC, and to define key residues in the Fc region that determine CDC activity of the bovine IgG subtypes. The assay utilizes CHO target cells engineered to express canine CD80 which binds to CTLA4 on the Fc fusion proteins. These target cells have been used in past canine ADCC assays and were utilized in the CDC assay due to their dependability.

For bIgG3a CTLA4 Fc fusions, the assay methods were performed as described in Examples above.

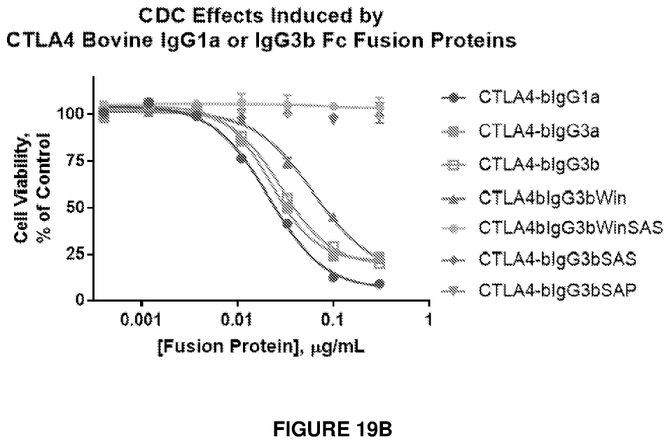

As shown in FIG. 19 and Table 12, the bIgG3a wildtype Fc and the bIgG3b wildtype Fc showed slightly less potent CDC activity than the bIgG1a wildtype Fc. The Win mutation by itself does not knockout CDC activity of bIgG3a Fc or bIgG3b Fc. The Win-SAS, SAS-only, or SAP-only mutations all completely knockout CDC activity.

### In Silico Modeling

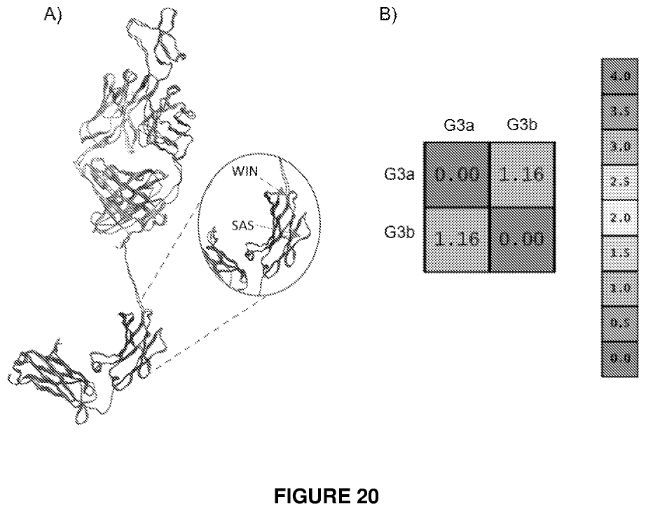

Like subclass IgG1, the effect of WINSAS mutations in bovine subclass 3 was studied using the same methodology as above and overlayed using the same protocol (FIG. 20A). The WINSAS mutations in bIgG3a and bIgG3b both completely abrogated CDC function as shown in FIG. 19. The in-silico modeling data showed that the two allotypes, bIgG3a_WINSAS and bIgG3b_WINSAS have RMSD of 1.16 Å and therefore are expected to have similar protein folds (FIG. 20B). Binding data listed in Table 11 and the in-silico modeling data in FIG. 20 indicate that bIgG3a_WINSAS and bIgG_3b fold and bind in an identical manner and therefore, based on both experimental and model results, we predict with a high degree of certainty that, in addition to CDC function, the bIgG3b_WINSAS construct would also eliminate ADCP and ADCC functions similar to bIgG3a_WINSAS.

### ADCP Assay:

The ADCP cell-based assay was developed and employed to characterize the effectiveness of the four CTLA4 bovine IgG3a Fc fusion proteins (FIG. 18) in mediating ADCP, and to define key residues in the Fc region that determine ADCP activity of the bovine IgG subtypes. The assay utilizes CHO target cells engineered to express canine CD80 which binds to CTLA4 on the Fc fusion proteins.

For this assay, incubation of the Fc fusion protein/target cell complexes with bovine alveolar macrophages can result in Fc binding to Fc gamma receptors on the macrophages, bridging the CHO target cells and the macrophage effectors and thereby initiating phagocytosis of the target cells. ADCP is measured by signal intensity and frequency of a pH-sensitive fluorescent dye within the population of effector macrophages in the co-culture, wherein fluorescent cells are indicative of an effector that has successfully internalized a target cell into the acidic lysosome.

Briefly, canine CD80-expressing CHO cells (CD80 target cells) or parental CHO cells not expressing CD80 (parental target cells) are pre-stained with pHrodo red dye for 30 minutes at 37 degrees C. Cells are then incubated with CTLA4-Fc fusion proteins for 20 minutes to mediate CTLA4:CD80 binding, and subsequently co-cultured with pre-plated adherent bovine alveolar macrophages stained with a cell marker (CellTrace Violet, CTV) to aid in later identification. 60,000 target cells are plated with 30,000 effector macrophages in a 96 well plate. Cells are co-cultured for 6 hours, and subsequently removed from plates and analyzed by flow cytometry to identify effector cells (CTV+) that have successfully performed ADCP (pHrodo+).

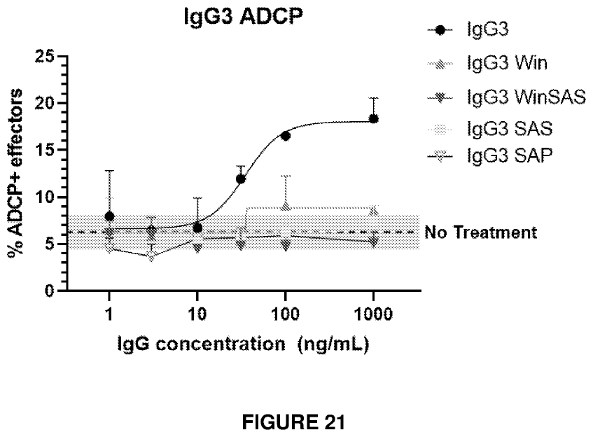

As shown in FIG. 21 and Table 12, wildtype bIgG3a is a potent activator of ADCP in bovine alveolar macrophages. The bIgG3aWin alone, WinSAS, SAS alone, and SAP alone mutations all eliminate ADCP function.

Notably, although the Win mutation of bIgG3a does show some trace activity at the highest concentrations, this mutation is largely ineffective at driving ADCP in contrast to its activity in both bIgG1a ADCP, as well as bIgG3a CDC. This may indicate that bIgG3a ADCP activity is only mediated through a subset of bFcgRs in alveolar macrophages which requires the Winter site mutation to bind, whereas bIgG1a mediates ADCP through a separate bFcgR that does not require the Winter site to bind in these cells. A variable utilization of FcgRs could also explain the different observed EC50s of the wildtype bIgG1a compared to bIgG3a. Conversely, divergent secondary structures between bIgG1a and bIgG3a may result in differential utilization of the Winter site in binding to the appropriate receptors driving ADCP.

TABLE 12
CDC and ADCP effects induced by bovine IgG3a
Fc wildtype, IgG3b Fc wildtype and mutations.

| CTLA4-bIgG3a Fusions | CDC EC50 Value | ADCP EC50 Value |
|---|---|---|
| CTLA4 Fc bIgG1a | 0.013 μg/mL | 0.003 μg/mL |
| wildtype (control) | | |
| CTLA4 Fc bIgG3a wildtype | 0.021 μg/mL | 0.029 μg/mL |
| CTLA4 Fc bIgG3aWin | 0.030 μg/mL | No ADCP |
| CTLA4 Fc bIgG3aWin-SAS | No CDC | No ADCP |
| CTLA4 Fc bIgG3aSAS | No CDC | No ADCP |
| CTLA4 Fc bIgG3aSAP | No CDC | |

| CTLA4-bIgG3b Fusions | CDC EC50 Value |
|---|---|
| CTLA4 Fc bIgG1a wildtype (control) | 0.011 μg/mL |
| CTLA4 Fc bIgG3a wildtype (control) | 0.022 μg/mL |
| CTLA4 Fc bIgG3b wildtype | 0.028 μg/mL |
| CTLA4 Fc bIgG3bWin | 0.062 μg/mL |
| CTLA4 Fc bIgG3bWin-SAS | No CDC |
| CTLA4 Fc bIgG3bSAS | No CDC |
| CTLA4 Fc bIgG3bSAP | No CDC |

### Example 9

### bIgG Mutation Improves FcRn Affinity

As shown in Table 13, bovine IgG3a has weak binding affinity to bFcRn at pH6 compared to bIgG2a. Unexpectedly, bIgG2a has a 10× higher affinity than bIgG1a and bIgG3a, although bIgG2a binding at pH7.4 to bovine FcRn is stronger when compared to the other two subclasses. However, tighter binding for bIgG2a at the pH required for releasing antibody back into circulation (pH7.4) is not a concern because of the demonstrated long serum half-life for bIgG2a-based mAbs.

A bIgG2-based mAb has a serum half-life in calves significantly longer than most human therapeutic mAbs, up to 21-days. Bovine IgG2a has stronger affinity to bFcRn at pH6 than bIgG1a does. Hence bIgG3a subclass may have a shorter serum half-life than bIgG2a. Thus, it is desirable to increase the affinity of bIgG3a to bFcRn to extend in vivo half-life comparable to bIgG2a.

TABLE 13
Binding affinity of bIgG3a wildtype
and bIgG3a_R433H mutant to bFcRn.

| | bFcRn, pH 6 | bFcRn, pH 7.4 |
|---|---|---|
| Bovine IgG mAbs | KD (M) | KD (M) |
| bIgG1a | 3.14E−08 | NBO |
| bIgG2a | 3.06E−09 | 1.31E−07 |
| bIgG3a | 3.02E−08 | NBO |
| bIgG3_R433H | 9.14E−09 | NBO |

NBO = No Binding Observed.

In order to improve affinity to bFcRn, a point mutation was introduced on bIgG3a (Arg433His). This mutation was based on alignment with bIgG1a and bIgG2a as shown in FIG. 2. In addition, a human IgG3 allotype with His435 has a dramatically longer serum half-life than the allotype with Arg435, and this residue position is in the same region as an arginine in bIgG3, just two residues upstream from the human arginine: HEALHNRY for hIgG3 and HEALRNHY for bIgG3 (FIG. 2).

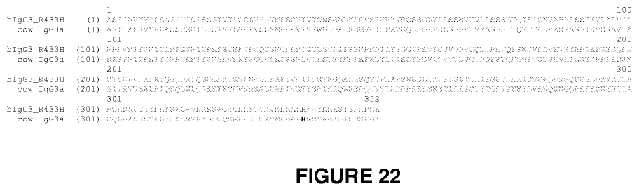

A R433H mutant bIgG3a mAb was generated for improved affinity to bFcRn, as described above. Alignment of the constant region of the bIgG3a wildtype recombinant mAb with the R433H mutation is shown in FIG. 22.

A Biacore SPR binding assay was designed to test the affinity of bovine IgG subclasses to bovine FcRn, as described in Examples above.

The bIgG3a_R433H mutant binds to bFcRn at pH6 with a 5× higher affinity than bIgG3a wildtype, and the mutant retains negligible binding to bFcRn at pH7.4 (Table 13). Thus, the R433H mutant could improve serum half-life.

Having described preferred embodiments of the invention, it is to be understood that the invention is not limited to the precise embodiments, and that various changes and modifications may be effected therein by those skilled in the art without departing from the scope or spirit of the invention as defined in the appended claims.

1-85. (canceled)

86. A modified IgG comprising: a bovine IgG constant domain comprising at least one amino acid substitution relative to a wild-type bovine IgG constant domain, wherein said substitution is at amino acid residue 216, 234, 235, 237, 270, 329, 330, 331, 432, 434, or 437, numbered according to the Eu index as in Kabat, and wherein said substitution is D216E, P234A, L235A, G237A, D270E, P329S, A330S, P331S, L432A, N434A, or T437A.

87. The modified IgG of claim 86, wherein the IgG constant domain comprises CH1 domain or a hinge region.

88. The modified IgG of claim 86, wherein the IgG constant domain comprises an Fc constant region having CH2 and CH3 domain.

89. A pharmaceutical composition comprising the modified IgG of claim 86 and a pharmaceutically acceptable carrier.

90. A kit comprising the modified IgG of claim 86, in a container, and instructions for use.

91. A vector comprising the nucleic acid sequence encoding any one of the amino acid sequences set forth in SEQ ID NOs.: 10-12, 14-25, and 27-31.

92. An isolated cell comprising the vector of claim 91.

93. A method of manufacturing an antibody or a molecule, the method comprising: providing the cell of claim 92; and culturing said cell.

94. A fusion molecule comprising: a bovine IgG constant domain fused to an agent, said bovine IgG constant domain comprising the modified IgG of claim 86.
